(19) United States
(12) Patent Application Publication
(10) Pub. No.: US 2023/0151270 A1
(43) Pub. Date: May 18, 2023

(54) Aggregation-Induced Emission Polymer, Preparation Method and Application Thereof

(71) Applicants: Qingdao Agricultural University, Qingdao City (CN); The Hong Kong University of Science and Technology, Hong Kong (CN)

(72) Inventors: Lei HAN, Qingdao City (CN); Benzhong TANG, Qingdao City (CN); Chenchen LIU, Qingdao City (CN); Xuhui BIAN, Qingdao City (CN)

(21) Appl. No.: 17/696,298

(22) Filed: Mar. 16, 2022

(30) Foreign Application Priority Data
Nov. 12, 2021 (CN) 202111341713.0

(51) Int. Cl. C09K 11/06; C08B 37/00; C12P 19/26

(52) U.S. Cl. CPC C09K 11/06; C08B 37/006; C12P 19/26; C09K 2211/145; C09K 2211/1425

(57) ABSTRACT

The present disclosure provides an aggregation-induced emission polymer, a preparation method and application thereof. The aggregation-induced emission polymer provided in the present disclosure has a structure represented by formula I. The aggregation-induced emission polymer provided by the present disclosure has excellent fluorescence stability and biocompatibility; Because there are many benzene rings in the aggregation-induced emission polymer, the fat-solubility of the aggregation-induced emission polymer is increased, thereby changing the problem that cellulose is insoluble and difficult to be processed and modified. In the present disclosure, the aggregation-induced emission small molecule monomer is placed in a basal medium, and the bacterial seed solution is inoculated and then cultured to obtain the aggregation-induced emission polymer. The preparation method provided by the present disclosure has the characteristics of safety, environmental protection and simplicity, solves the shortcomings of complex and cumbersome synthesis process, and is beneficial to the large-scale production of AIE polymers.

### CROSS REFERENCE TO RELATED APPLICATION

This patent application claims the benefit and priority of Chinese Patent Application No. 202111341713.0, filed on Nov. 12, 2021, the disclosure of which is incorporated by reference herein in its entirety as part of the present application.

### TECHNICAL FIELD

The present disclosure relates to the technical field of polymer materials, in particular to an aggregation-induced emission polymer, a preparation method and application thereof.

### BACKGROUND ART

In 2001, Academician Tang Benzhong et al. discovered that silacyclopentadiene (silole) did not emit light in solution but emitted strong fluorescence in the aggregation state (nanoparticles in poor solvents or thin films in solid state), and defined this phenomenon as aggregation-induced emission (AIE). AIE molecules overcome the shortcomings of aggregation-caused quenching (ACQ) molecules. At present, polymers containing functional groups with AIE properties (such as tetrastyrene, triphenylamine, etc.) (hereinafter referred to as AIE polymers) have broad application prospects in the fields of organic light-emitting diodes, biological imaging, fluorescent probes (such as detection of heavy metal ions, explosives, and pH, etc.) and biological probes (such as detection of DNA, RNA and protein, etc.). However, the currently reported AIE polymers are all polymerized from monomers through chemical methods.

Macromolecule bacterial cellulose (BC) with the structure represented by formula III is a common biopolymer, but the polymer has weak fluorescence or no fluorescence.

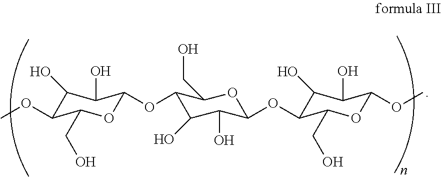

### SUMMARY

In view of this, the purpose of the present disclosure is to provide an aggregation-induced emission polymer and a preparation method and application thereof. The aggregation-induced emission polymer provided by the present disclosure has excellent fluorescence stability.

In order to achieve the above purpose of the present disclosure, the present disclosure provides the following technical schemes:

The present disclosure provides an aggregation-induced emission polymer, wherein having a structure represented by formula I:

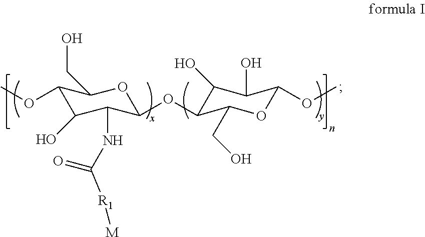

In the formula I, R₁ includes any one of the following structures:

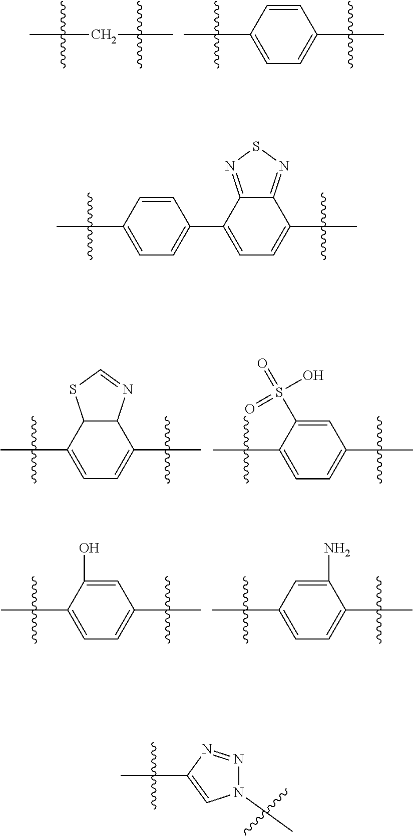

In the formula I, M has the structure represented by formula M-a or formula M-b:

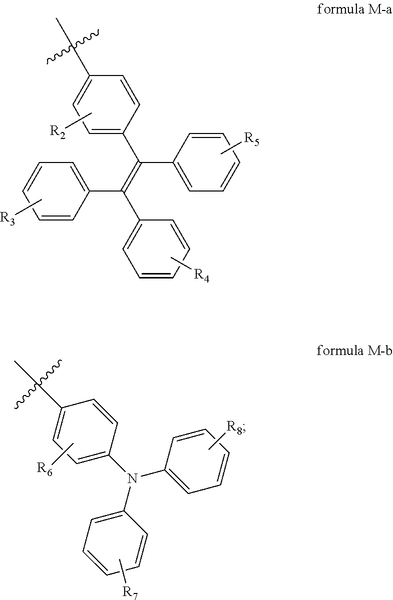

R₂-R₅ in the formula M-a and R₆-R₈ in the formula M-b independently include any one of the following structures:

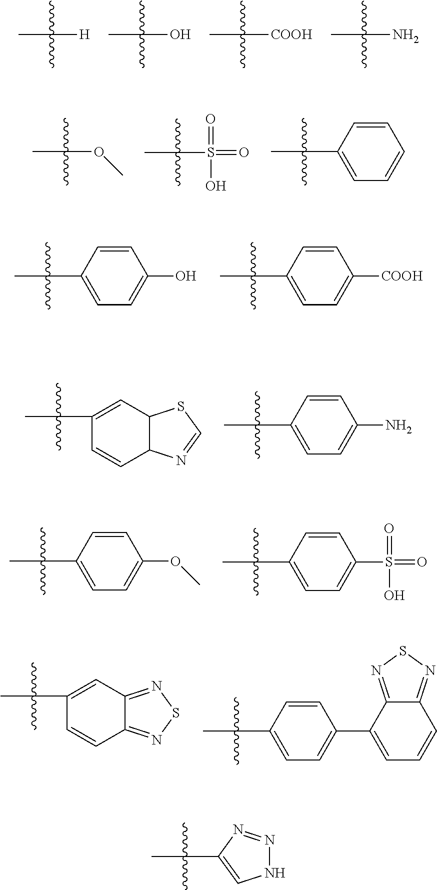

n is 1500-6000.

The present disclosure provides a method for preparing the aggregation-induced emission polymer described in above technical schemes, wherein comprising the following steps:

Placing an aggregation-induced emission small molecule monomer in a basal medium, then inoculating a bacterial seed solution, and culturing the obtained reaction solution to obtain an aggregation-induced emission polymer;

The aggregation-induced emission small molecule monomer has a structure represented by formula II:

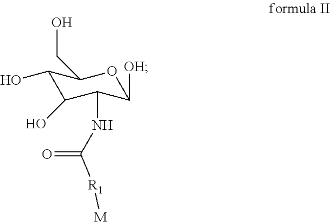

In the formula II, R₁ includes any one of the following structures:

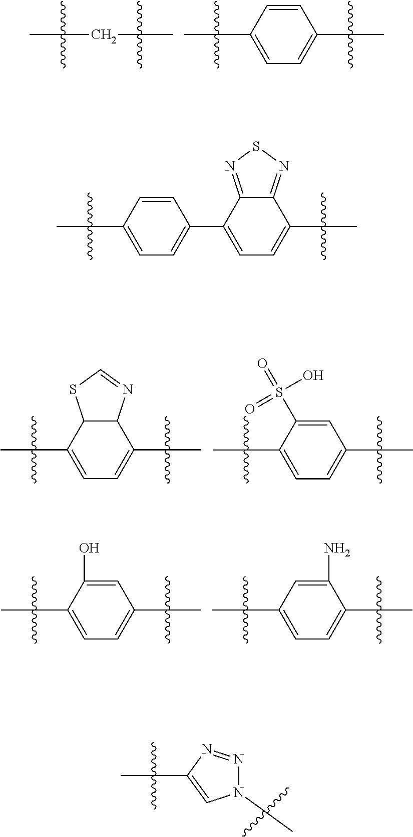

In the formula II, M has the structure represented by formula M-a or formula M-b:

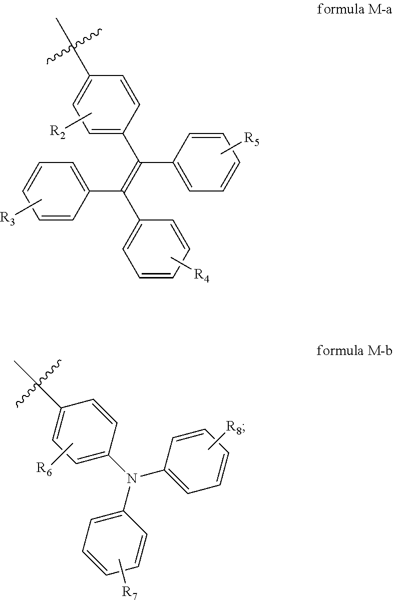

R₂-R₅ in the formula M-a and R₆-R₈ in the formula M-b independently include any one of the following structures:

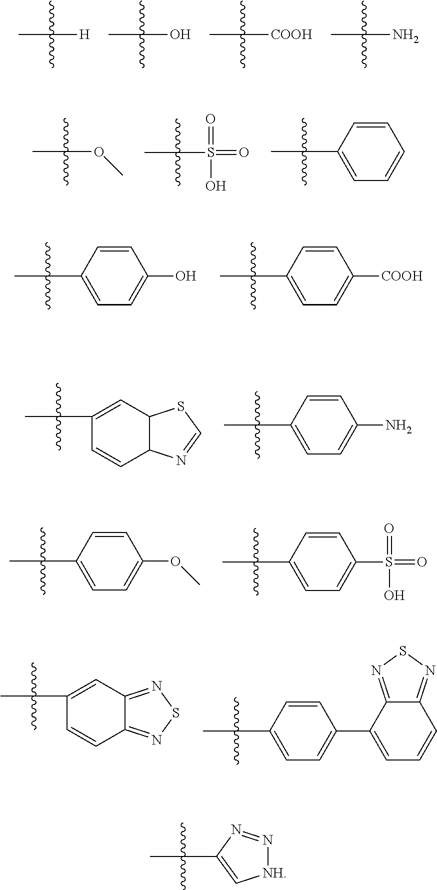

In some embodiments, the aggregation-induced emission small molecule monomer preferably has a structure represented by formula IIa, formula IIb or formula IIc:

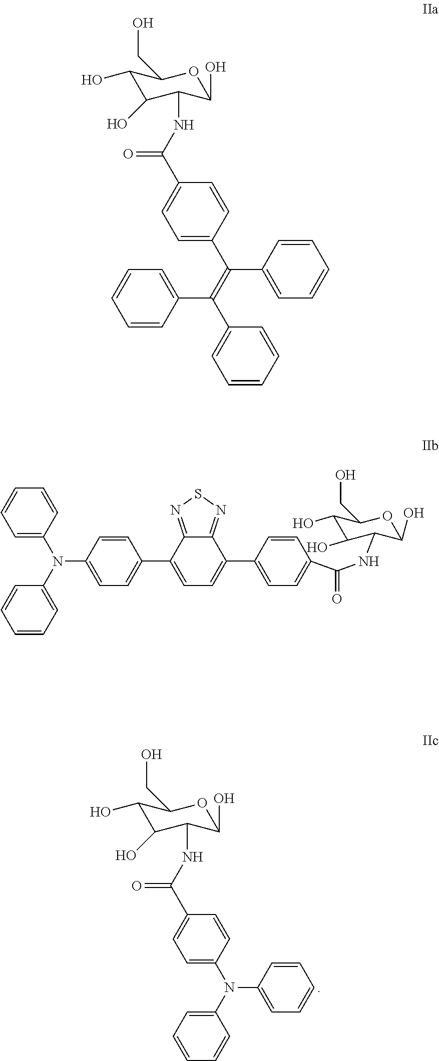 [SMILES: O=C(NC1C(O)OC(CO)C(O)C1O)c1ccc(C(=C(c2ccccc2)c2ccccc2)c2ccccc2)cc1 ; O=C(NC1C(O)OC(CO)C(O)C1O)c1ccc(-c2ccc(-c3ccc(N(c4ccccc4)c4ccccc4)cc3)c3nsnc23)cc1 ; O=C(NC1C(O)OC(CO)C(O)C1O)c1ccc(N(c2ccccc2)c2ccccc2)cc1]

In some embodiments, the concentration of the aggregation-induced emission small molecule monomer in the reaction solution is 0.001-1 mg/mL.

In some embodiments, the chemical composition of the basal medium comprises: 20-30 g/L glucose, 4-6 g/L yeast extract, 4-6 g/L peptone, 1.1-1.3 g/L citric acid, 2.3-2.9 g/L disodium hydrogen phosphate and water.

In some embodiments, the inoculum size of the bacterial seed solution is 1-50% of the volume of the medium; and the bacterial cell density (OD₆₀₀) of the bacterial seed solution is 0.6-1.2.

In some embodiments, the temperature of the culture is 20-45° C., and the time is 2-8 d.

The present disclosure provides the application of the aggregation-induced emission polymer described in above technical schemes or the aggregation-induced emission polymer prepared by the preparation method described in above technical schemes in light-emitting diodes, bioimaging, fluorescent films, biosensors or chiral separations.

The present disclosure provides an aggregation-induced emission polymer (AIE polymer), which has a structure represented by formula I. The aggregation-induced emission polymer provided by the present disclosure does not cause fluorescence quenching due to the aggregation of π-π C stacking, and has excellent fluorescence stability, large Stokes shift, large fluorescence intensity, and large quantum yield; Because there are many benzene rings in the aggregation-induced emission polymer, the fat-solubility of the aggregation-induced emission polymer is increased, thereby changing the problem that cellulose is insoluble and difficult to be processed and modified; bacterial cellulose (BC) and its derivatives are polymers produced by microorganisms with high biocompatibility.

The aggregation-induced emission polymer provided by the present disclosure can be modified with triazole groups, tetraphenylethylene (TPE), phosphoric acid groups and other functional groups with metal ion and biomolecule detection capabilities. Among them, the triazole group can be combined with mercury ions to form a complex to quench the fluorescence, thereby detecting mercury ions; Phosphate group modified tetraphenylethylene (TPE) can be used for fluorescence detection of alkaline phosphatase, phosphate group increases the water solubility of small molecules, alkaline phosphatase makes it hydrolyze into poorly water-soluble AIE polymer to detect alkaline phosphatase using aggregation-induced emission. Therefore, the aggregation-induced emission polymer provided by the present disclosure can be used as an ideal polymer for biochemical analysis and fluorescent probes.

Fluorescent materials used on organic light emitting diodes (OLED) or polymer light emitting diodes (PLED) are usually in a solid or thin film state. Compared with some traditional emission materials, the aggregation-induced emission polymer provided by the present disclosure can avoid the ACQ (aggregation-caused quenching) effect of traditional emission materials in solid state, and has the advantages of good fluorescence stability, high quantum yield, and electroluminescence, which can be applied to the production of OLED and PLED, and because BC is degradable, it has a greater application prospect.

The aggregation-induced emission polymer provided by the present disclosure can generate ROS (reactive oxygen species) under light, and the ROS will cause serious damage to the cell to achieve the antibacterial effect, and can be used for antibacterial.

The aggregation-induced emission polymer provided by the present disclosure has AIE functional groups, the fluorescence is more stable, it is not easy to be quenched, and has good long-term tracing capability that a good polymer for long-acting imaging of cells should have, moreover, it is not easily degraded; BC is produced by bacteria, has good biocompatibility and a relatively stable structure, which is not easily degraded in the body. Therefore, the aggregation-induced emission polymer prepared by the present disclosure can be used as an ideal polymer for long-term biological imaging.

As chiral recognition materials, cellulose and cellulose derivatives have been widely used in chiral separations. This is because they have a regular and ordered supramolecular structure, and there are a large number of chiral cavities and chiral sites inside the molecule. When the racemate passes through, there is a certain difference in the spatial matching degree of the chiral cavity formed by the enantiomeric molecule and the polar group, and the resulting force is different. The aggregation-induced emission polymer provided by the present disclosure is a cellulose modified with aromatic molecules. The present disclosure can enhance the solubility and chiral recognition ability of cellulose by modifying aromatic molecules on cellulose, and is an ideal chiral separation material.

The present disclosure provides a method for preparing the aggregation-induced emission polymer described in the above technical scheme. Compared with the traditional organic synthesis method of aggregation-induced emission polymer, the present disclosure synthesizes the aggregation-induced emission polymer by one-step biosynthesis method which not require multi-step reaction and purification steps, has the characteristics of safety, environmental protection and simplicity, solves the shortcomings of complex and tedious synthesis process, and is beneficial to the large-scale production of AIE polymer compounds. The aggregation-induced emission polymer synthesized through bacterial synthesis of the present invention has high biocompatibility. The preparation method provided by the present invention is versatile and is suitable for the biosynthesis of aggregation-induced emission polymers from glucose monomers modified by common AIE small molecules.

### BRIEF DESCRIPTION OF THE DRAWINGS

FIG. 1 shows the infrared spectra of TPE-BC and BC;

FIG. 2 is a picture of TPE-BC and BC under sunlight and 365 nm ultraviolet light;

FIG. 3 shows the infrared spectra of TB-BC and BC;

FIG. 4 is a picture of TB-BC and BC under sunlight and 365 nm ultraviolet light;

FIG. 5 is a CLSM diagram of time monitoring TB-BC synthesis;

FIG. 6 is a picture of TB-BC/polyvinylpyrrolidone (PVP) and PVP electrospun film under sunlight and 365 nm ultraviolet light;

FIG. 7 is the fluorescence excitation and emission spectra of TPE-BC;

FIG. 8 is the fluorescence excitation and emission spectra of TB-BC;

FIG. 9 is the fluorescence excitation and emission spectra of 6CF-BC;

FIG. 10 shows the fluorescence spectra of TPE-BC and BC;

FIG. 11 shows the fluorescence spectra of TB-BC and BC;

FIG. 12 is a CLSM diagram of BC, 5CF-BC and TPE-BC;

FIG. 13 is a CLSM diagram of BC, TB/BC and TB-BC;

FIG. 14 is a scanning electron microscope (SEM) image of TB-BC.

### DETAILED DESCRIPTION OF THE EMBODIMENTS

The present disclosure provides an aggregation-induced emission polymer, wherein having a structure represented by formula I:

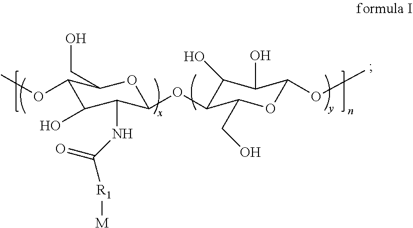

In the present disclosure, in formula I, R₁ includes any one of the following structures:

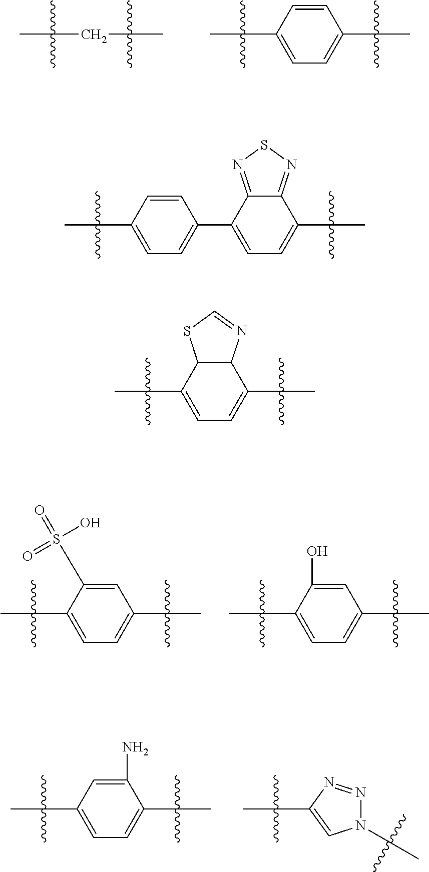

In the present disclosure, in formula I, M has the structure represented by formula M-a or formula M-b:

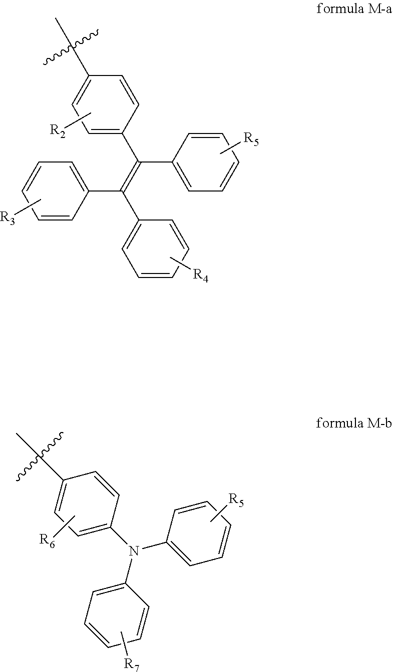

In the present disclosure, R₂-R₅ in the formula M-a and R₆-R₈ in the formula M-b independently include any one of the following structures:

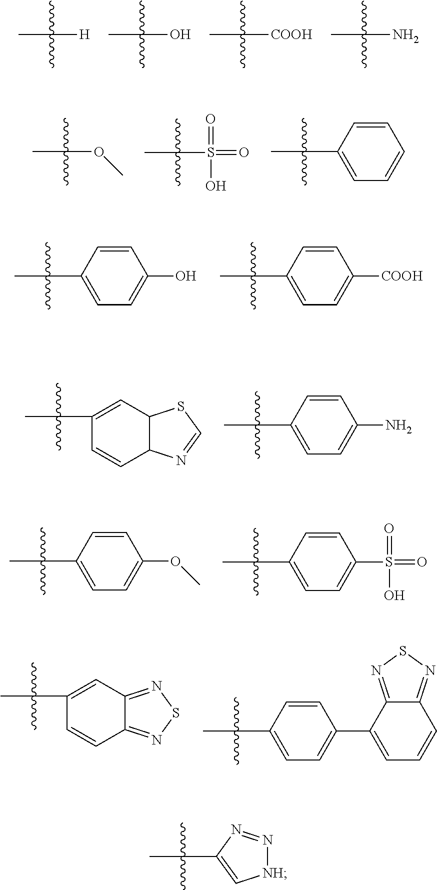

In the present disclosure, n is 1500-6000, preferably 2000-5000, and more preferably 3000-4000.

In the present disclosure, the aggregation-induced emission polymer preferably has any one of the structures represented by formulas I-1 to 1-3:

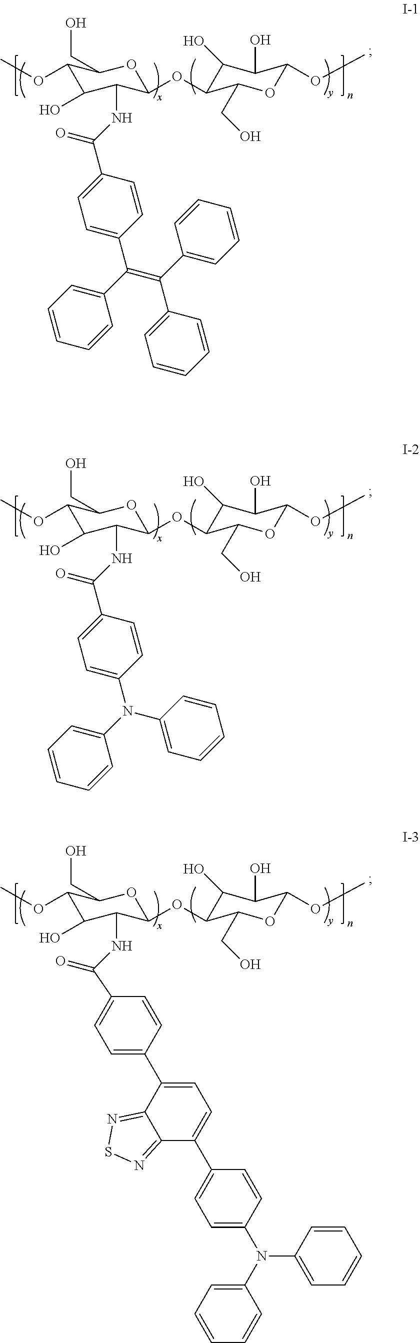

The present disclosure provides a method for preparing the aggregation-induced emission polymer described in above technical schemes, wherein comprising the following steps:

Placing an aggregation-induced emission small molecule monomer in a basal medium, then inoculating a bacterial seed solution, and culturing the obtained reaction solution to obtain an aggregation-induced emission polymer;

The aggregation-induced emission small molecule monomer has a structure represented by formula II:

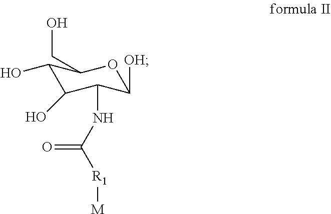

In the present disclosure, unless otherwise specified, all raw material components are commercially available products well known to those skilled in the art.

In the present disclosure, the optional groups of R₁ and M in the formula II are preferably the same as the optional groups of R₁ and M in the formula I, and will not be repeated here.

In the present disclosure, the aggregation-induced emission small molecule monomer preferably has a structure represented by formula IIa, formula IIb or formula IIc:

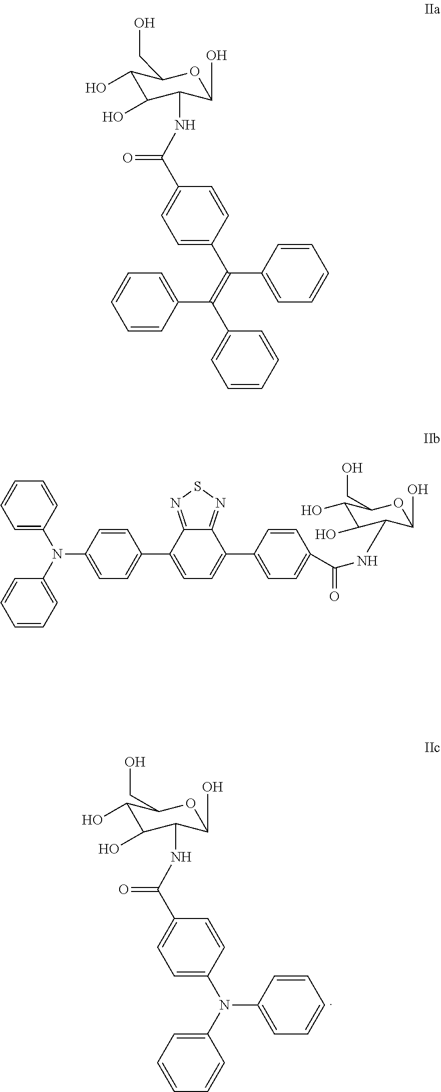 [SMILES: O=C(NC1C(O)OC(CO)C(O)C1O)c1ccc(C(=C(c2ccccc2)c2ccccc2)c2ccccc2)cc1 ; O=C(NC1C(O)OC(CO)C(O)C1O)c1ccc(-c2ccc(-c3ccc(N(c4ccccc4)c4ccccc4)cc3)c3nsnc23)cc1 ; O=C(NC1C(O)OC(CO)C(O)C1O)c1ccc(N(c2ccccc2)c2ccccc2)cc1]

In the present disclosure, the preparation route of the aggregation-induced emission small molecule monomer having the structure represented by formula IIa is shown in formula (1), and the specific steps are as follows:

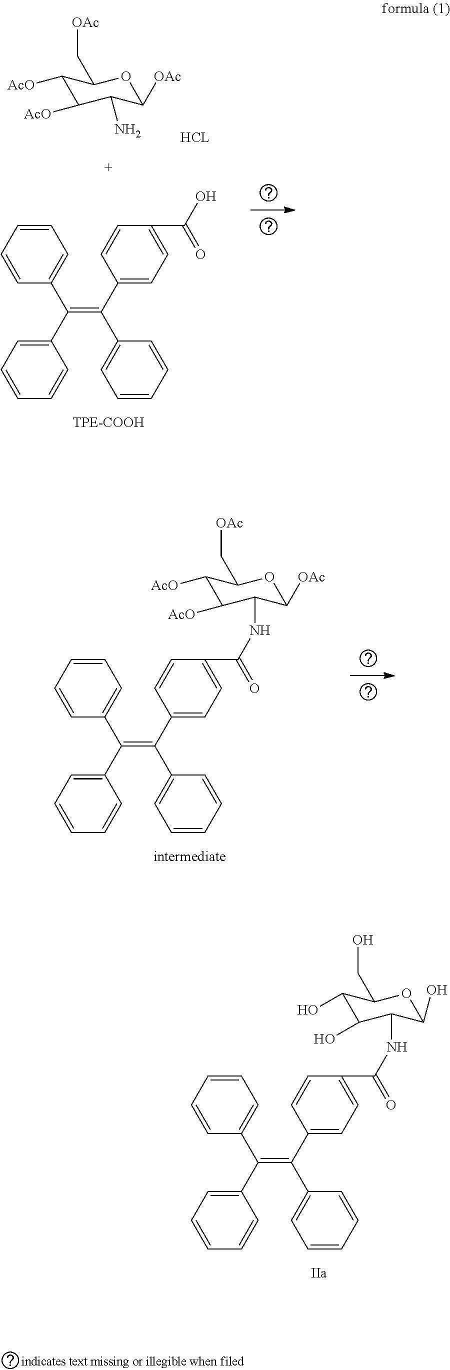 [SMILES: CC(=O)OCC1OC(OC(C)=O)C(N)C(OC(C)=O)C1OC(C)=O ; O=C(O)c1ccc(C(=C(c2ccccc2)c2ccccc2)c2ccccc2)cc1 ; CC(=O)OCC1OC(OC(C)=O)C(NC(=O)c2ccc(C(=C(c3ccccc3)c3ccccc3)c3ccccc3)cc2)C(OC(C)=O)C1OC(C)=O ; O=C(NC1C(O)OC(CO)C(O)C1O)c1ccc(C(=C(c2ccccc2)c2ccccc2)c2ccccc2)cc1]

Mixing the compound TPE-COOH, N, N, N′, N′-tetramethyl-O—(N-succinimidyl) uronium tetrafluoroborate (TSTU), N, N-diisopropylethylamine (DIPEA) and an organic solvent, and incubating to obtain an activated TPE-COOH solution;

Mixing the activated TPE-COOH solution and 1,3,4,6-tetra-O-acetyl-2-amino-2-deoxy-β-D-glucopyranose to perform an amidation reaction to obtain an intermediate;

Subjecting the intermediate to a deacetylation reaction under alkaline conditions to obtain an aggregation-induced emission small molecule monomer having a structure represented by formula IIa.

In the present disclosure, the compounds TPE-COOH, N, N, N′, N′-tetramethyl-O—(N-succinimidyl) uronium tetrafluoroborate, N, N-diisopropylethylamine and the organic solvents are mixed and activated to obtain an activated TPE-COOH solution. In the present disclosure, the mass ratio of the compound TPE-COOH, TSTU and DIPEA is preferably 400:400-550:400-650, more preferably 400:410-500:450-620, and further preferably 400:415:592. In the present disclosure, the organic solvent preferably includes N, N-dimethylformamide (DMF), tetrahydrofuran (THF), dimethyl sulfoxide (DMSO); In the present disclosure, the amount of the organic solvent is not particularly limited, as long as the activation can proceed smoothly; in the embodiment of the present disclosure, the mass ratio of the compound TPE-COOH to the volume ratio of the organic solvent is preferably 1 g:50 mL. The present disclosure has no particular limitation on the mixing, as long as the raw materials can be mixed uniformly. In the present disclosure, the temperature of the incubation is preferably 15-30° C., more preferably room temperature, the time of the incubation is preferably 10-60 min, more preferably 20-50 min, further preferably 30 min; the incubation is preferably carried out under protective atmosphere conditions, the protective atmosphere preferably includes nitrogen or inert gas, and the inert gas preferably includes helium or argon; during the incubation process, the N-succinimide group is combined with the carboxyl group to become an active state.

After obtaining the activated TPE-COOH solution, the present disclosure mixes the activated TPE-COOH solution with 1,3,4,6-tetra-O-acetyl-2-amino-2-deoxy-β-D-glucopyranose, and carries out cultivation to obtain intermediates.

In the present disclosure, the 1,3,4,6-tetra-O-acetyl-2-amino-2-deoxy-β-D-glucopyranose is preferably used in the form of a 1,3,4,6-tetra-O-acetyl-2-amino-2-deoxy-β-D-glucopyranose solution, the concentration of the 1,3,4,6-tetra-O-acetyl-2-amino-2-deoxy-β-D-glucopyranose solution is preferably 10-50 g/L, more preferably 20-40 g/L, and further preferably 26.5 g/L. The solvent in the 1,3,4,6-tetra-O-acetyl-2-amino-2-deoxy-β-D-glucopyranose solution preferably includes DMF, THF, or DMSO. In the present disclosure, the mass ratio of the compound TPE-COOH and 1,3,4,6-tetra-O-acetyl-2-amino-2-deoxy-β-D-glucopyranose is preferably 40: 40-70, more preferably 40: 50-60, further preferably 40: 53. The present disclosure has no particular limitation on the mixing, as long as the raw materials can be mixed uniformly. In the present disclosure, the temperature of the amidation reaction is preferably 15-30° C., more preferably room temperature, and the time of the amidation reaction is preferably 8-16 h, more preferably 10-14 h, and further preferably 12 h; The amidation reaction is preferably carried out under light-shielding and protective atmosphere conditions. The protective atmosphere preferably includes nitrogen or an inert gas, and the inert gas preferably includes helium or argon.

After the amidation reaction, the present disclosure preferably further includes purifying the reaction solution of the amidation reaction to obtain an intermediate. In the present disclosure, the purification includes silica gel column chromatography separation and thin-layer chromatography separation in sequence; The eluent used in the silica gel column chromatography separation is preferably a dichloromethane-methanol mixed solvent, and the volume ratio of dichloromethane-methanol in the dichloromethane-methanol mixed solvent is preferably 5-15 1, more preferably 10:1; the developing agent used for the thin-layer chromatography separation is preferably a dichloromethane-methanol mixed solvent, and the volume ratio of dichloromethane-methanol in the dichloromethane-methanol mixed solvent is preferably 5-15:1, more preferably 10:1.

After the intermediate is obtained, in the present disclosure, the intermediate is subjected to a deacetylation reaction under alkaline conditions to obtain an aggregation-induced emission small molecule monomer having a structure represented by formula IIa. In the present disclosure, the alkaline conditions are preferably provided by an alkaline solution, and the alkaline solution preferably includes a NaOH solution or a KOH solution; the concentration of the alkaline solution is preferably 0.005-0.2 mol/L, more preferably 0.01-0.15 mol/L, further preferably 0.1-0.15 mol/L; the solvent in the alkali solution preferably includes an alcohol solvent-water mixed solvent, and the volume ratio of the alcohol solvent to water in the alcohol solvent-water mixed solvent is preferably 0.5-5:1, more preferably 1-3:1, further preferably 1:1; the alcohol solvent preferably includes methanol, ethanol, n-butanol or isopropanol. In the present disclosure, the mixing is preferably ultrasonic mixing, the ultrasonic power of the ultrasonic mixing is preferably 100-400 W, more preferably 200-300 W; the ultrasonic mixing time is preferably 1-10 s, more preferably 2-8 s, further preferably 5 s. In the present disclosure, the temperature of the deacetylation reaction is preferably 15-50° C., more preferably room temperature, the deacetylation reaction is preferably monitored by thin layer chromatography; the time of the deacetylation reaction is preferably 5-30 min, more preferably 10-20 min.

After the deacetylation reaction is completed, the present disclosure preferably further includes a post-treatment, the post-treatment includes: adjusting the pH value of the reaction solution of the deacetylation reaction to 7, performing a first concentrating, extracting the obtained concentrated liquid, and successively subjecting the obtained organic phase to drying, a second concentrating and purification by silica gel chromatography to obtain the aggregation-induced emission small molecule monomer having the structure represented by formula IIa. In the present disclosure, the acid used for the pH adjustment is preferably hydrochloric acid, and the concentration of the hydrochloric acid is preferably 0.005-0.2 mol/L, more preferably 0.01-0.1 mol/L, and further preferably 0.05 mol/L. In the present disclosure, the method of first concentration is preferably rotary evaporation, and the temperature of the rotary evaporation is preferably 40-90° C., more preferably 50-70° C.; the purpose of the first concentration is to remove alcohol solvents. In the present disclosure, the extractant used in the extraction preferably includes dichloromethane (DCM) and ethyl acetate; the number of the extraction is preferably 3-4 times. In the present disclosure, the method of drying is preferably drying with a desiccant, and the desiccant is preferably anhydrous magnesium sulfate. In the present disclosure, there is no special limitation on the method of the second concentration, as long as the concentration method well known to those skilled in the art can be used, specifically, such as vacuum distillation. In the present disclosure, the eluent used in the silica gel chromatographic purification preferably includes a dichloromethane-methanol mixed solvent, and the volume ratio of dichloromethane to methanol in the dichloromethane-methanol mixed solvent is preferably 2-10:1, more preferably 3-8:1, further preferably 5:1.

In the present disclosure, the preparation route of the aggregation-induced emission small molecule monomer having the structure represented by formula IIb is shown in formula (2), and the specific steps are as follows:

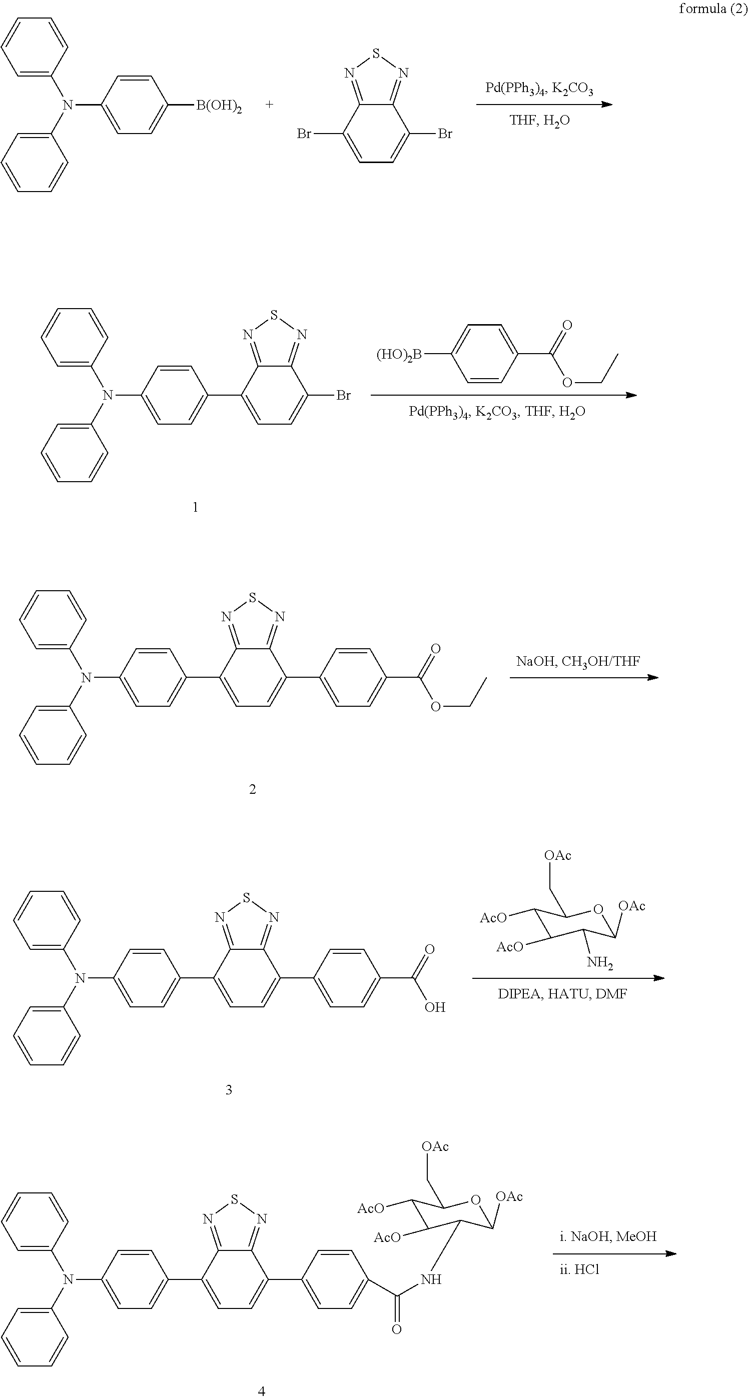

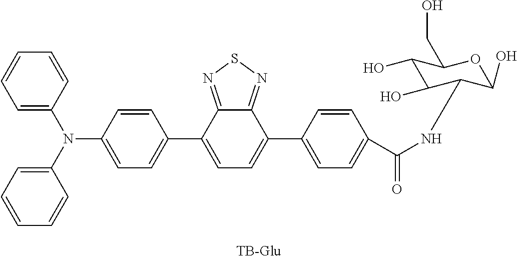 [SMILES: O=C(NC1C(O)OC(CO)C(O)C1O)c1ccc(-c2ccc(-c3ccc(N(c4ccccc4)c4ccccc4)cc3)c3nsnc23)cc1]

Mixing compound 1, (4-(ethoxycarbonyl) phenyl) boronic acid, Pb(PPh₃)₄, K₂CO₃ aqueous solution and an organic solvent, and performing a coupling reaction to obtain compound 2;

Subjecting the compound 2 to a hydrolysis reaction under alkaline conditions to obtain compound 3;

Mixing the compound 3, 1,3,4,6-tetra-O-acetyl-B-D-glucosamine, 2-(7-azabenzotriazol-1-yl)-N, N, N′, N′-tetramethyluronium hexafluorophosphate (HATU), DIPEA and an organic solvent, and performing an amidation reaction to obtain compound 4;

Subjecting the compound 4 to a deacetylation reaction under alkaline conditions to obtain an aggregation-induced emission small molecule monomer having a structure represented by formula IIb.

In the present disclosure, compound 1, (4-(ethoxycarbonyl) phenyl) boronic acid, Pb(PPh₃)₄, K₂CO₃ aqueous solution and organic solvent are mixed to carry out substitution reaction to obtain compound 2. In the present disclosure, the molar ratio of the compound 1, (4-(ethoxycarbonyl) phenyl) boronic acid, Pb(PPh₃)₄ and K₂CO₃ in the K₂CO₃ aqueous solution is preferably 1:0.9-1.2:0.02-0.03:0.005-0.015, more preferably 1:1 0.026:0.01; the concentration of the K₂CO₃ aqueous solution is preferably 1-5 mol/L, more preferably 2 mol/L. In the present disclosure, the organic solvent preferably includes tetrahydrofuran, N,N-dimethylformamide or dimethyl sulfoxide, and the volume ratio of tetrahydrofuran and water in the mixed solvent is preferably 8-15:1, more preferably is 12:1; the present disclosure has no special limitation on the amount of the organic solvent, as long as it can ensure the smooth progress of the coupling reaction; in the embodiment of the present disclosure, the ratio of the amount of the compound 1 and the volume of the organic solvent is preferably 1 mmol: 10-15 mL, and more preferably 1 mmol: 12 mL. The present disclosure has no particular limitation on the mixing, as long as the raw materials can be mixed uniformly. In the present disclosure, the temperature of the coupling reaction is preferably 50-100° C., more preferably 80° C., the time of the coupling reaction is preferably 12-36 h, more preferably 24 h; the coupling reaction is preferably carried out under a protective atmosphere, and the protective atmosphere preferably includes nitrogen or an inert gas, and the inert gas preferably includes helium or argon. After the coupling reaction, the present disclosure preferably further includes post-treatment, the post-treatment includes: cooling the reaction solution of the coupling reaction to room temperature and then extracting, and sequentially subjecting the obtained organic phase to drying, concentrating and purifying by silica gel chromatography to obtain compound 2; the present disclosure has no special limitation on the cooling method, as long as it is cooled to room temperature; the extractant for extraction preferably includes dichloromethane or ethyl acetate; the number of extractions is preferably 3-4 times. The drying method is preferably desiccant drying, and the desiccant is preferably anhydrous magnesium sulfate; the present disclosure has no particular limitation on the concentration method, and the concentration method well known to those skilled in the art can be used, and the specific example is vacuum distillation; the eluent used in the silica gel chromatography purification preferably includes a hexane-ethyl acetate mixed solvent, and the volume ratio of hexane and ethyl acetate in the hexane-ethyl acetate mixed solvent is preferably 1-8:1, more preferably 2-6:1, further preferably 3:1.

After compound 2 is obtained, the present disclosure subjects the compound 2 to a hydrolysis reaction under alkaline conditions to obtain a compound 3. In the present disclosure, the alkaline condition is preferably provided by an inorganic base, the inorganic base preferably includes NaOH or KOH; the mass ratio of the compound 2 to the inorganic base is preferably 1-3:1, more preferably 1.5-2.5:1, more preferably 2.625:1. In the present disclosure, the organic solvent for the hydrolysis reaction preferably includes a methanol-tetrahydrofuran mixed solvent, and the volume ratio of methanol to tetrahydrofuran in the methanol-tetrahydrofuran mixed solvent is preferably 1:0.5-2, more preferably 1:1-1.5; The present disclosure does not specifically limit the amount of the organic solvent, as long as it can ensure the smooth progress of the hydrolysis reaction; in the embodiment of the present disclosure, the ratio of the mass of the compound 2 to the volume of the organic solvent is preferably 1 g: 40-50 mL, more preferably 1 g: 46-47 mL. In the present disclosure, the temperature of the hydrolysis reaction is preferably 50-100° C., more preferably 80° C., and the time of the hydrolysis reaction is preferably 8-16 h, more preferably 10-12 h. After the hydrolysis reaction, the present disclosure preferably further includes post-treatment, the post-treatment includes: cooling the reaction solution of the hydrolysis reaction to room temperature and then extracting, and then sequentially subjecting the obtained organic phase to drying, concentrating and purifying by silica gel chromatography to obtain the compound 3; the present disclosure has no special limitation on the cooling method, as long as it is cooled to room temperature; the extractant for extraction preferably includes dichloromethane or ethyl acetate; the number of extractions is preferably 3-4 times; The drying method is preferably desiccant drying, and the desiccant is preferably anhydrous magnesium sulfate; the present disclosure has no special limitation on the concentration method, and the concentration method well-known to those skilled in the art may be used, such as vacuum distillation; The eluent used in the silica gel chromatography purification preferably includes a hexane-ethyl acetate mixed solvent, and the volume ratio of hexane and ethyl acetate in the hexane-ethyl acetate mixed solvent is preferably 1:2-10, more preferably 1:4.

After compound 3 is obtained, the compound 3, 1,3,4,6-tetra-O-acetyl-B-D-glucosamine, 2-(7-azabenzotriazol-1-yl)-N, N, N′, N′-tetramethyluronium hexafluorophosphate (HATU), N, N-diisopropylethylamine (DIPEA) and an organic solvent are mixed, and a substitution reaction is performed to obtain compound 4. In the present disclosure, the mass ratio of compound 3, 1,3,4,6-tetra-O-acetyl-B-D-glucosamine, 2-(7-azabenzotriazol-1-yl)-N, N, N′, N′-tetramethyluronium hexafluorophosphate and N, N-diisopropylethylamine is preferably 1:0.8-0.85:0.9-0.92:1.2-1.6, more preferably 1:0.832:0.913:1.48. In the present disclosure, the organic solvent preferably includes DMF, THF, and DMSO; in the present disclosure, the amount of the organic solvent is not particularly limited, as long as it can ensure the smooth progress of the amidation reaction; in the embodiment of the present disclosure, the ratio of the mass of the compound 3 to the volume of the organic solvent is preferably 1 g: 80-120 mL, and more preferably 1 g: 100 mL. The present disclosure has no particular limitation on the mixing, as long as the raw materials can be mixed uniformly. In the present disclosure, the temperature of the amidation reaction is preferably 100-130° C., more preferably 120° C., the time of the amidation reaction is preferably 12-36 h, more preferably 24 h; the amidation reaction is preferably carried out under a protective atmosphere, and the protective atmosphere preferably includes nitrogen or an inert gas, and the inert gas preferably includes helium or argon. After the amidation reaction, the present disclosure preferably further includes a post-treatment. The post-treatment includes: cooling the reaction solution of the amidation reaction to room temperature and then extracting, and sequentially subjecting the obtained organic phase to drying, concentrating and purifying by silica gel chromatography to obtain compound 4; the present disclosure has no special limitation on the cooling method, as long as it is cooled to room temperature; the extractant for extraction preferably includes dichloromethane or ethyl acetate; the number of extractions is preferably 3-4 times. The drying method is preferably desiccant drying, and the desiccant is preferably anhydrous magnesium sulfate; the present disclosure has no particular limitation on the concentration method, and the concentration method well known to those skilled in the art can be used, specifically such as vacuum distillation; the eluent used in the silica gel chromatography purification preferably includes a hexane-ethyl acetate mixed solvent, and the volume ratio of hexane and ethyl acetate in the hexane-ethyl acetate mixed solvent is preferably 1-3:1, more preferably 1-2:1.

After the compound 4 is obtained, the present disclosure performs a deacetylation reaction of the compound 4 under alkaline conditions to obtain an aggregation-induced emission small molecule monomer having a structure represented by formula IIb. In the present disclosure, the alkaline condition is preferably provided by an alkaline solution, the alkaline solution preferably includes NaOH solution and/or KOH solution; the concentration of the alkaline solution is preferably 0.05-0.5 mol/L, more preferably 0.1-0.3 mol/L; the solvent in the alkali solution preferably includes an alcohol solvent-water mixed solvent, and the volume ratio of the alcohol solvent to water in the alcohol solvent-water mixed solvent is preferably 0.5-2:1, more preferably 1:1; the alcohol solvent preferably includes methanol, ethanol, n-butanol or isopropanol. The present disclosure has no particular limitation on the mixing, as long as the raw materials can be mixed uniformly. In the present disclosure, the temperature of the deacetylation reaction is preferably 15-30° C., more preferably room temperature, the deacetylation reaction is preferably monitored by thin layer chromatography; the time of the deacetylation reaction is preferably 1-3 h, more preferably 2-2.5 h. After the deacetylation reaction is completed, the present disclosure preferably further includes a post-treatment, and the post-treatment includes: adjusting the pH value of the reaction solution of the deacetylation reaction to 7, performing extracting, and sequentially subjecting the obtained organic phase to drying, concentrating and purifying by silica gel chromatography to obtain the aggregation-induced emission small molecule monomer with the structure represented by formula IIb. In the present disclosure, the acid used for adjusting the pH value is preferably hydrochloric acid, and the concentration of the hydrochloric acid is preferably 0.05-0.5 mol/L, more preferably 0.1-0.3 mol/L. In the present disclosure, the extractant used in the extraction preferably includes dichloromethane (DCM) or ethyl acetate; the number of extraction is preferably 3-4 times. In the present disclosure, the drying method is preferably drying with a desiccant, and the desiccant is preferably anhydrous magnesium sulfate. In the present disclosure, there is no particular limitation on the method of concentration, and a concentration method well known to those skilled in the art may be used, such as vacuum distillation. In the present disclosure, the eluent used in the silica gel chromatographic purification preferably includes a dichloromethane-methanol mixed solvent, and the volume ratio of dichloromethane and methanol in the dichloromethane-methanol mixed solvent is preferably 3-8:1, more preferably 5-6:1.

In the present disclosure, the aggregation-induced emission small molecule monomer is preferably used in the form of an solution of the aggregation-induced emission small molecule monomer, and the concentration of the solution of the aggregation-induced emission small molecule monomer is preferably 5-40 μg/mL, more preferably 10-30 μg/mL, further preferably 20 μg/mL; the solvent in the solution of the aggregation-induced emission small molecule monomer preferably includes one or more of dimethyl sulfoxide, tetrahydrofuran, dichloromethane, N,N-dimethylformamide and ethylene glycol.

In the present disclosure, the chemical composition of the basal medium preferably comprises: 20-30 g/L glucose, 4-6 g/L yeast extract, 4-6 g/L peptone, 1.1-1.3 g/L citric acid, 2.3-2.9 g/L disodium hydrogen phosphate and water. In the basal medium, the concentration of glucose is more preferably 23-28 g/L, and further preferably 25 g/L; the concentration of yeast extract is more preferably 4.5-5.5 g/L, and further preferably 5 g/L; the concentration of the peptone is more preferably 4.5-5.5 g/L, further preferably 5 g/L; the concentration of the citric acid is more preferably 1.15-1.25 g/L, further preferably 1.2 g/L; the concentration of the disodium hydrogen phosphate is more preferably 2.4-2.8 g/L, further preferably 2.5 g/L; and the water is preferably deionized water. In the present disclosure, the basal medium is preferably sterilized before being used and then cooled to room temperature; the temperature of the sterilization is preferably 80-130° C., more preferably 90-120° C., and further preferably 100-100° C.; the time of the sterilization treatment is preferably 10-40 min, more preferably 15-35 min, further preferably 20-30 min; the present disclosure has no special limitation on the cooling method, and the cooling method well known to those skilled in the art can be used.

In the present disclosure, the bacterial species in the bacterial seed solution include Acetobacter xylinus, Gluconacetobacter xylinus, Achromobacter, Agrobacterium, Aerobacter, Azotobacter or Rhizobium; the OD₆₀₀ of the bacterial seed solution is preferably 0.5-1.2, more preferably 0.6-1.0; The inoculum size of the bacterial seed solution is preferably 1-50% of the volume of the medium, more preferably 10-40%, and further preferably 20-30%. In the present disclosure, the bacterial seed solution is preferably obtained by inoculating bacterial species into a basal medium for cultivation; the volume of the basal medium is preferably 5-25 mL, more preferably 10-20 mL; the basal medium is preferably the same as the aforementioned basal medium, which will not be repeated here; the basal medium is preferably sterilized before use, and the sterilization treatment is preferably the same as the aforementioned sterilization treatment, which will not be repeated here; the culture temperature is preferably 20-45° C., more preferably 30-40° C.; the culture time is preferably 10-24 h, more preferably 15-20 h.

In the present disclosure, the concentration of the aggregation-induced emission small molecule monomer in the reaction solution is preferably 0.001-1 mg/mL, more preferably 0.01-0.8 mg/mL, and further preferably 0.1-0.5 mg/mL.

In the present disclosure, the temperature of the culture is preferably 20-45° C., more preferably 25-40° C., and further preferably 30-35° C.; the culture time is preferably 2-8 d, more preferably 4-6 d, further preferably 5 d; the cultivation is preferably carried out in a constant temperature incubator. In the present disclosure, taking the AIE monomer having the structure represented by formula M-a as an example, during the culture process, the AIE monomer is phosphorylated by glucokinase to obtain a-6-phosphate, which is further converted into a-1-phosphate by the isomerization of phosphoglucose isomerase, glucose pyrophosphorylase is converted into uridine diphosphate-a, and uridine diphosphate-a is connected by β-1,4-glycosidic bond to synthesize AIE polymer. In the present disclosure, the synthesis of the aggregation-induced emission polymer during the cultivation process is preferably monitored by a confocal laser microscope (CLSM), so as to realize the visual monitoring of the production process of the aggregation-induced emission polymer.

After the culturing, the present disclosure preferably further includes subjecting the cultured system to post-treatment, and the post-treatment includes sequentially performing a first water washing, an alkali treatment, a second water washing and drying to obtain aggregation-induced emission polymers. In the present disclosure, the first water washing is preferably rinsing with distilled water. The present disclosure has no particular limitation on the number of the first water washing, as long as the basal medium and impurities on the surface can be removed. In the present disclosure, the alkali treatment is preferably performed with an alkaline reagent solution, and the concentration of the alkaline reagent solution is preferably 0.1-1 mol/L, more preferably 0.5-0.8 mol/L; The alkaline reagent in the alkaline reagent solution is preferably a hydroxide, and the hydroxide preferably includes sodium hydroxide and/or potassium hydroxide; the temperature of the alkali treatment is preferably 25-90° C., more preferably 40-80° C., and further preferably 50-60° C.; the time of the alkali treatment is preferably 3-20 h, more preferably 5-15 h, further preferably 10-12 h; the purpose of the alkali treatment is to remove bacterial protein and residual basal medium; The alkali treatment preferably further includes cooling to room temperature; the present disclosure has no special limitation on the cooling method, and the cooling method well known to those skilled in the art may be used. In the present disclosure, the second water washing is preferably distilled water washing; in the present disclosure, there is no special limitation on the number of the second water washing, and it is sufficient to wash with water until the liquid is neutral. In the present disclosure, the drying method is preferably vacuum drying; the drying temperature is preferably 20-50° C., more preferably 30-40° C.; the present disclosure has no particular limitation on the drying time, as long as it is dried to constant weight.

Traditional AIE polymers are prepared by organic synthesis methods, one method is that AIE monomers and non-AIE monomers are chemically polymerized to form AIE polymers. The synthesis process usually has the following shortcomings: The use of organic solvents such as N, N-dimethylformamide and triphenylamine can cause serious negative impacts on the environment, increase the complexity of waste disposal, and restricte its large-scale production; reaction conditions often require deoxygenation, dehumidification, and organic solvents, the reaction conditions are relatively high; at the same time, it also affects the biocompatibility of AIE polymers; organically synthesized AIE polymers have disadvantages such as low yield and complex purification process. The present disclosure synthesizes the aggregation-induced emission polymer through the biosynthesis method without multi-step reaction and purification steps, has the characteristics of safety, environmental protection and simplicity, solves the shortcomings of complex and cumbersome synthesis process, and is beneficial to the large-scale production of AIE polymer compounds. The preparation method provided by the disclosure is synthesized by bacterial synthesis, and the obtained aggregation-induced emission polymer has high biocompatibility. The preparation method provided by the present disclosure is versatile and is suitable for the biosynthesis of aggregation-induced emission polymers from glucose monomers modified by common AIE small molecules.

The present disclosure provides applications of the aggregation-induced emission polymer described in the above technical scheme or the aggregation-induced emission polymer obtained by the preparation method described in the above technical scheme in light-emitting diodes, biological imaging, fluorescent films, biosensors or chiral separation.

Fluorescent materials used on organic light emitting diodes (OLED) or polymer light emitting diodes (PLED) are usually in a solid or thin film state. Compared with some traditional emission materials, the aggregation-induced emission polymer provided by the present disclosure can avoid the ACQ (aggregation-caused quenching) effect of traditional fluorescent materials in solid state, and has the advantages of good fluorescence stability, high quantum yield, and electroluminescence, which can be applied to the production of OLED and PLED, and because BC is degradable, it has a greater application prospect.

The aggregation-induced emission polymer prepared by the present disclosure can be modified with triazole groups, phosphoric acid groups and other functional groups capable of detecting metal ions and biomolecules. Among them, the triazole group can be combined with mercury ions to form a complex to quench the fluorescence to detect mercury ions; Phosphate group-modified TPE can be used for fluorescent detection of alkaline phosphatase, phosphate group increases the water solubility of small molecules, alkaline phosphatase makes it hydrolyze into poorly water-soluble AIE polymer to detect alkaline phosphatase using aggregation-induced emission, therefore, the aggregation-induced emission polymer provided by the present disclosure can be used as an ideal polymer for biochemical analysis and fluorescent probes.

The aggregation-induced emission polymer provided by the present disclosure can generate ROS (reactive oxygen species) under light, and the ROS will cause serious damage to the cell to achieve the antibacterial effect, and can be used for antibacterial.

The aggregation-induced emission polymer provided by the present disclosure has AIE functional group, the fluorescence is more stable, it is not easy to be quenched, and has good long-term tracing capability that a good polymer for long-acting imaging of cells should have, moreover it is not easily degraded; BC is produced by bacteria, has good biocompatibility and a relatively stable structure, and is not easily degraded in the body. Therefore, the aggregation-induced emission polymer prepared by the present disclosure can be used as an ideal polymer for long-term biological imaging.

As chiral recognition materials, cellulose and cellulose derivatives have been widely used in chiral separations. This is because they have a regular and ordered supramolecular structure, and there are a large number of chiral cavities and chiral sites inside the molecule. When the racemate passes through, there is a certain difference in the spatial matching degree of the chiral cavity formed by the enantiomeric molecule and the polar group, and the resulting force is different. The aggregation-induced emission polymer prepared by the present disclosure is a cellulose modified with aromatic molecules. The present disclosure can enhance the solubility and chiral recognition ability of cellulose by modifying aromatic molecules on cellulose, and is an ideal chiral separation material. Moreover, the preparation method provided by the present disclosure has better solubility than chemical modification, the preparation process is simpler, and the modification does not need to be modified by a solid-liquid heterogeneous reaction.

The technical schemes of the present disclosure will be clearly and completely described below in conjunction with the embodiments of the present disclosure. Obviously, the described embodiments are only a part of the embodiments of the present disclosure, rather than all the embodiments. Based on the embodiments of the present disclosure, all other embodiments obtained by those of ordinary skill in the art without creative work shall fall within the protection scope of the present disclosure.

### Example 1

According to the reaction route shown in formula (1), the aggregation-induced emission small molecule monomer (TPE-Glu) with the structure represented by formula IIa is prepared, and the specific steps are as follows: The compound TPE-COOH (400 mg) was dissolved in dry N, N-dimethylformamide (20 mL), N, N, N′, N′-tetramethyl-O—(N-succinimidyl) uronium tetrafluoroborate (TSTU) (415 mg) and N, N-diisopropylethylamine (DIPEA, 0.8 mL) were added, the mixture was mixed, and incubated at room temperature under inert gas protection for 30 min to obtain an activated TPE-COOH solution. The 1,3,4,6-tetra-O-acetyl-2-amino-2-deoxy-β-D-glucopyranose solution (530 mg, 20 mL) was mixed with the activated TPE-COOH solution, subjected to amidation reaction at room temperature under inert gas protection and dark conditions for 12 h, the reaction product was separated and purified by silica gel column chromatography and thin layer chromatography (eluent and developing solvent were dichloromethane/MeOH, 10:1, v/v) to obtain the intermediate (white powder, a yield of 73%, a purity of 99%).

The intermediate (53 mg) was added to a NaOH solution (0.01 mol/L, 3 mL, MeOH/H₂O=1:1), subjected to ultrasonic treatment under 200 W at room temperature for 5 s, then deacetylated at room temperature for 10 min, the degree of deacetylation was monitored using thin-layer chromatography (developing solvent: dichloromethane/methanol=10:1), after the deacetylation reaction was completed, the pH value was adjusted to 7.0 using hydrochloric acid (3 mL, 0.01 mol/L), the methanol in the solution was removed by rotary evaporation at 50° C. for 10 min, the reaction mixture was extracted for three times with DCM (50 mL), the organic layer was dried with MgSO₄ and concentrated, the concentrate was purified by silica gel chromatography using DCM/MeOH (5:1, v/v) as the eluent, and vacuum dried for 24 h to obtain TPE-Glu (white powder, a yield of 67%, a purity of 99%).

Structural characterization of TPE-Glu: ¹H NMR (500 MHz, CDCl₃), δ(ppm): 7.86 (d, J=8.2 Hz, 2H), 7.06-7.19 (m, 12H), 6.94-7.05 (m, 5H), 4.50 (s, 1H), 3.47 (s, 1H), 2.81-2.97 (m, 5H).

### Example 2

According to the reaction route shown in formula (2), the aggregation-induced emission small molecule monomer (TPE-Glu) with the structure represented by formula IIb is prepared, and the specific steps are as follows:

The synthesis of compound 2: Under N₂ protection, compound 1 (2.29 g, 5 mmol), (4-(ethoxycarbonyl) phenyl) boronic acid (970 mg, 5 mmol), and Pb(PPh₃)₄ (30 mg, 0.026 mmol) were dissolved in THF (60 mL) and K₂CO₃ aqueous solution (2 mol/L, 5 mL), the mixture was heated to 80° C. and the coupling reaction was carried out under stirring for 24 h. After cooling to room temperature, it was extracted for three times with DCM, and then the organic phase was dried with anhydrous MgSO₄ and concentrated. Using hexane/ethyl acetate (3:1, v/v) as the eluent, the obtained concentrate was purified by silica gel chromatography to obtain compound 2 (yellow powder, a yield of 84%, and a purity of 99%). Structure characterization of compound 2: ¹H NMR (400 MHz, Chloroform-d) δ8.24-8.18 (m, 2H), 8.08-8.03 (m, 2H), 7.91-7.86 (m, 2H), 7.84-7.75 (m, 2H), 7.30 (dd, J=8.5, 7.2 Hz, 4H), 7.24-7.17 (m, 6H), 7.11-7.04 (m, 2H), 4.43 (q, J=7.1 Hz, 2H), 1.43 (t, J=7.1 Hz, 3H). ¹³C NMR (101 MHz, Chloroform-d) δ166.46, 154.07, 154.00, 148.32, 147.43, 141.83, 133.76, 131.40, 130.55, 130.03, 129.84, 129.42, 129.12, 128.79, 127.10, 125.03, 123.47, 122.73, 61.07, 14.40. HRMS (MALDI-TOF, m/z): [M] calcd for C₃₃H₂₅N₃O₂S 527.1667, found 527.1671.

Synthesis of compound 3 (abbreviated as TB): Compound 2 (1.05 g) and NaOH (0.4 g) were added to CH₃OH (25 mL) and THF (25 mL), and then the mixture was heated to 80° C. and stirred for 12 h. After that, the reaction mixture was cooled to room temperature and extracted for three times with DCM, and then the organic layer was dried with MgSO₄ and concentrated. Using hexane/ethyl acetate (1:4, v/v) as the eluent, the obtained concentrate was purified by silica gel chromatography to obtain compound 3 (yellow powder, a yield of 88%, a purity of 99%). Structure characterization of compound 3: ¹H NMR (400 MHz, Chloroform-d) δ8.30 (d, J=8.1 Hz, 2H), 8.13 (d, J=8.1 Hz, 2H), 7.92 (d, J=8.3 Hz, 2H), 7.88 (d, J=7.4 Hz, 1H), 7.82 (d, J=7.3 Hz, 1H), 7.33 (t, J=7.7 Hz, 4H), 7.24 (t, J=8.5 Hz, 6H), 7.11 (t, J=7.3 Hz, 2H). HRMS (MALDI-TOF, m/z): [M] calcd for C₃₁H₂₁N₃O₂S 499.1354, found 499.1355.

Synthesis of compound 4: Under N₂ protection, compound 3 (499 mg), 1,3,4,6-tetra-O-acetyl-B-D-glucosamine (English name (2S,3R,4S,5S,6R)-6-(acetoxymethyl)-3-aminotetrahydro-2H-pyran-2,4,5-triyl triacetate, 416 mg) and TSTU (361.32 mg) were dissolved in DMF (50 mL), heated to 120° C. and subjected to amidation reaction for 24 h under stirring conditions, after cooling to room temperature, it was extracted for three times with DCM, and then the organic phase was dried over anhydrous MgSO₄ and concentrated; Using hexane/ethyl acetate (1:1, v/v) as eluent, the obtained concentrate was purified by silica gel chromatography to obtain compound 4 (yellow powder, a yield of 73%, a purity of 99%). Structure characterization of compound 4: ¹H NMR (400 MHz, Chloroform-d) δ7.98 (d, J=8.1 Hz, 2H), 7.86 (dd, J=11.9, 8.3 Hz, 4H), 7.70 (s, 2H), 7.30 (t, J=7.7 Hz, 5H), 7.20 (t, J=6.3 Hz, 6H), 7.08 (t, J=7.3 Hz, 2H), 6.62 (d, J=9.5 Hz, 1H), 5.86 (d, J=8.8 Hz, 1H), 5.40 (t, J=10.1 Hz, 1H), 5.26 (t, J=9.7 Hz, 1H), 4.66 (q, J=9.6 Hz, 1H), 4.33 (dd, J=12.5, 4.7 Hz, 1H), 4.19 (dd, J=12.5, 2.3 Hz, 1H), 3.93 (ddd, J=10.0, 4.9, 2.2 Hz, 1H), 2.14-2.08 (m, 9H), 2.06 (s, 3H). ¹³C NMR (101 MHz, Chloroform-d) δ171.74, 170.73, 169.32, 166.95, 148.35, 147.39, 133.76, 132.89, 130.95, 130.00, 129.48, 129.42, 128.62, 127.28, 126.98, 125.05, 123.49, 122.66, 92.86, 73.20, 72.86, 67.86, 61.82, 53.33, 20.92, 20.77, 20.73, 20.61. HRMS (MALDI-TOF, m/z): [M] calcd for C₄₅H₄₀N₄O₁₀S 828.2465, found 828.2461.

Synthesis of TB-Glu (Formula IIb): NaOH solution (80 mg) and compound 4 (165.64 mg, 0.2 mmol) were added to MeOH (30 mL). Then the deacetylation reaction was carried out for 2 h under stirring at room temperature, and the degree of deacetylation was monitored by thin layer chromatography (developing solvent: dichloromethane/methanol, 10:1, v/v). After the deacetylation reaction was completed, the pH value was adjusted to 7.0 using hydrochloric acid (0.1 mol/L), the reaction mixture was extracted for three times with DCM (50 mL), the organic layer was dried with MgSO₄ and concentrated, using DCM/MeOH (5:1, v/v) as the eluent, and concentrated, the obtained concentrate was purified by silica gel chromatography, rotary evaporated at 50° C. and vacuum dried at 37° C. for 24 h to obtain TB-Glu (yellow powder, a yield of 53%, a purity of 99%).

Structure characterization of TB-Glu: ¹H NMR (400 MHz, DMSO-d₆) δ8.18-8.07 (m, 4H), 8.06-7.95 (m, 4H), 7.37 (dd, J=8.7, 7.1 Hz, 4H), 7.13 (dd, J=7.8, 2.3 Hz, 8H), 6.55 (dd, J=41.2, 5.4 Hz, 1H), 5.13 (t, J=3.9 Hz, 1H), 4.98 (dd, J=10.6, 5.4 Hz, 1H), 4.71 (dd, J=35.7, 6.3 Hz, 1H), 4.52 (dt, J=37.2, 5.8 Hz, 1H), 4.11 (q, J=5.2 Hz, 1H), 3.90-3.70 (m, 2H), 3.68-3.65 (m, 1H), 3.53 (dt, J=11.9, 6.0 Hz, 1H), 3.22 (d, J=5.3 Hz, 1H), 3.17 (d, J=5.2 Hz, 1H). ¹³C NMR (101 MHz, DMSO-d₆) δ166.58, 153.83, 148.09, 147.35, 139.86, 132.82, 131.12, 130.76, 130.73, 130.19, 129.38, 129.22, 128.21, 128.06, 127.81, 125.04, 124.11, 122.73, 90.94, 72.65, 71.54, 70.58, 61.64, 55.94. HRMS (MALDI-TOF, m/z): [M] calcd for C₃₇H₃₂N₄O₆S 660.2043, found 660.2054.

### Example 3

The basal medium was sterilized at 115° C. for 20-30 min and cooled to room temperature to obtain a sterilized basal medium; the composition of the basal medium was: 25 g/L glucose, 5 g/L yeast extract, 5 g/L peptone, 1.2 g/L citric acid, 2.7 g/L disodium hydrogen phosphate and deionized water.

A single colony of Acetobacter xylinum was inoculated into 20 mL of sterilized basal medium and cultured at 30° C. for 24 h to obtain the Acetobacter xylinum seed solution.

The aggregation-induced emission small molecule with the structure represented by TPE-Glu (IIa) was dissolved in dimethyl sulfoxide to obtain an aggregation-induced emission small molecule solution with a concentration of 10 μg/mL.

The aggregation-induced emission small molecule solution was added to the sterilized basal medium and mixed evenly, the Acetobacter xylinum seed solution accounting for 7% of the volume of the basal medium was inoculated, and cultivated in a constant temperature incubator at 30° C. for 4-5 d. After rinsing with distilled water to remove the basal medium and impurities on the surface, it was placed in a sodium hydroxide solution with a concentration of 0.5 mol/L and subjected to alkali treatment at 60° C. for 12 h to remove bacterial protein and residual basal medium, cooled to room temperature, fully washed with distilled water until the pH of the washing solution was neutral, and vacuum dried to constant weight at 30° C. to obtain aggregation-induced emission polymer, i.e. BC modified with TPE (abbreviated as TPE-BC).

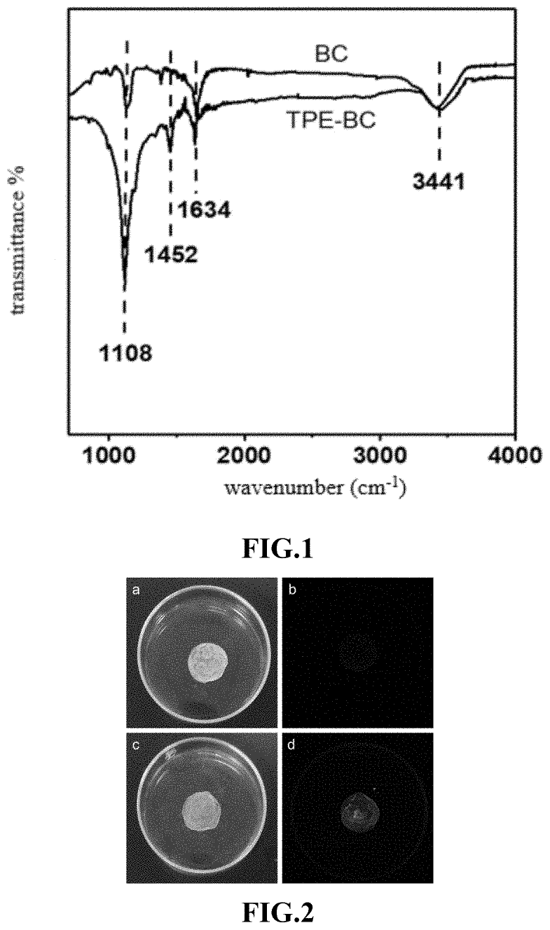

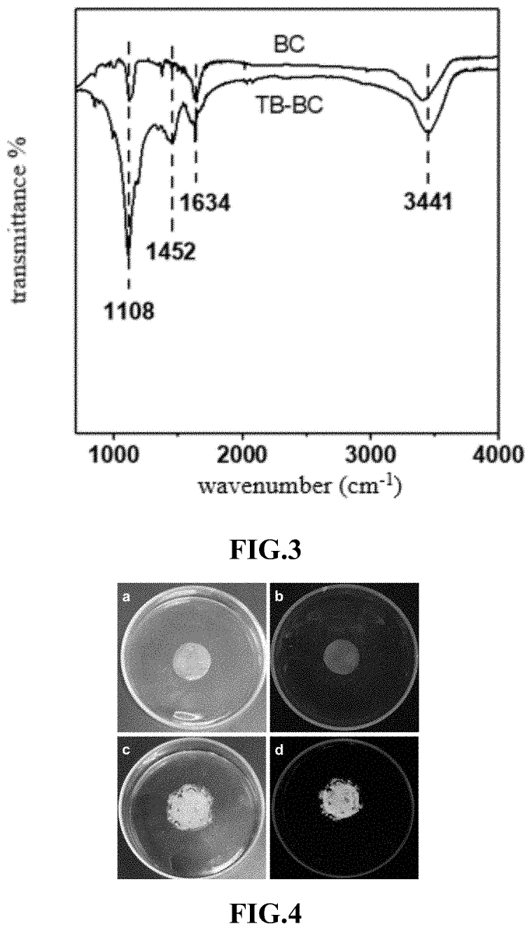

FIG. 1 shows the infrared spectra of TPE-BC and BC. As can be seen from FIG. 1, a represents TPE-BC and b represents BC. It can be seen from FIG. 3 that TPE-BC has an ether bond stretching peak at 1108 cm⁻¹, a C═O characteristic peak at 1452 cm⁻¹, a C═N characteristic peak at 1650 cm⁻¹, and a OH stretch peak at 3441 cm⁻¹; BC has characteristic peaks at 1108 cm⁻¹, 1650 cm⁻¹, 3441 cm⁻¹, but no peak at 1452 cm⁻¹, indicating that the present disclosure prepares the aggregation-induced emission polymer TPE-BC with the above structure.

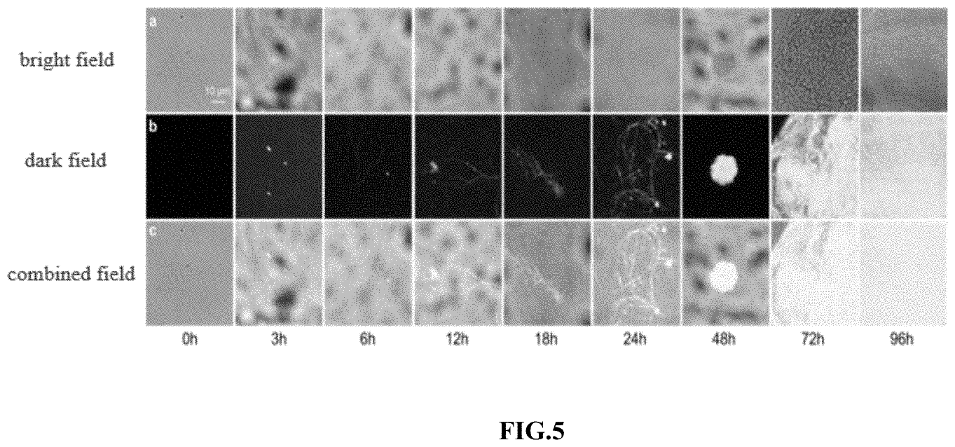

FIG. 2 is a picture of TPE-BC and BC under sunlight and 365 nm ultraviolet light, where a and b are Comparative Example 1, and c and d are Example 3. It can be seen from FIG. 4 that the synthesized product of Example 3 emits bright cyan-blue fluorescence under 365 nm ultraviolet light, and BC has no obvious fluorescence, indicating that the TPE-BC prepared by the present disclosure is an aggregation-induced emission polymer with fluorescence properties can be applied to fluorescent films.

### Example 4

The aggregation-induced emission polymer was prepared according to the method of Example 3. The difference from Example 3 is that using the aggregation-induced emission small molecule has the structure represented by formula IIb to obtain the aggregation-induced emission polymer, i.e., the BC molecule modified with TB (abbreviated as TB-BC).

FIG. 3 shows the picture of TB-BC and BC under sunlight and 365 nm ultraviolet light, wherein a represents TB-BC and b represents BC. It can be seen from FIG. 3 that TB-BC has an ether bond stretching peak at 1108 cm⁻¹, a C═O characteristic peak at 1452 cm⁻¹, a C═N characteristic peak at 1650 cm⁻¹, and a OH stretch peak at 3441 cm⁻¹; BC has characteristic peaks at 1108 cm⁻¹, 1650 cm⁻¹, 3441 cm⁻¹, but no peak at 1452 cm⁻¹, indicating that the present disclosure prepares the aggregation-induced emission polymer TB-BC with the above structure.

FIG. 4 is a picture of TB-BC and BC under sunlight and 365 nm ultraviolet light, where a and b are BC, and c and d are TB-BC. It can be seen from FIG. 4 that the synthesized product of TBG-BC emits bright orange-yellow fluorescence under 365 nm ultraviolet light, and BC has no obvious fluorescence, indicating that the TB-BC prepared by the present disclosure is an aggregation-induced emission polymer with fluorescence properties which can be applied to fluorescent films.

### Example 5

The aggregation-induced emission polymer was prepared according to the method of Example 4. The difference from Example 4 is that the static culture time is different. They were cultured for 0, 3, 6, 12, 18, 24, 48, 72, and 96 h respectively, then inspected by laser confocal microscope, the inspection result is shown in FIG. 5. It can be seen from FIG. 5 that after 3 h of culture, Acetobacter xylinum was adhered with the aggregation-induced emission small molecules. After 12 h of culture, aggregation-induced emission fibers were produced, the cultivation time was increased, and the agglomeration-induced emission fibers were increased and gradually aggregated.

### Example 6

2 g of polyvinylpyrrolidone (K-30) was dissolved in 4 mL of absolute ethanol to obtain a K-30 solution; 2 mg of TB-BC prepared in Example 3 was dissolved in 1 mL of tetrahydrofuran to obtain a TB-BC solution; the TB-BC solution was added to the K-30 solution and stirred for 30 min to obtain an electrostatic spinning solution. Electrospinning was carried out on the electrospinning instrument, the electrospinning solution was put into a 25 mL syringe, and then pumped into the nozzle at a propulsion speed of 0.005 mm/s, a positive voltage of 10 kV was applied to the electrospinning solution through the needle of a stainless steel syringe, the distance between the needle tip and the collector was kept at 10-15 cm, and the resulted products were collected on the aluminum foil to obtain the TB-BC/PVP electrospun polymer fiber membrane.

### Comparative Example 1

2 g of polyvinylpyrrolidone (K-30) was dissolved in 5 mL of absolute ethanol to obtain a K-30 solution; Electrospinning was carried out on the electrospinning instrument, the K-30 solution was put into a 25 mL syringe, and then pumped into the nozzle at a propulsion speed of 0.0051 mm/s, a positive voltage (10 kV) was applied to the polymer solution through the needle of a stainless steel syringe, the distance between the needle tip and the collector was kept at 10-15 cm, and the resulted products were collected on the aluminum foil to obtain the PVP electrospun polymer fiber membrane.

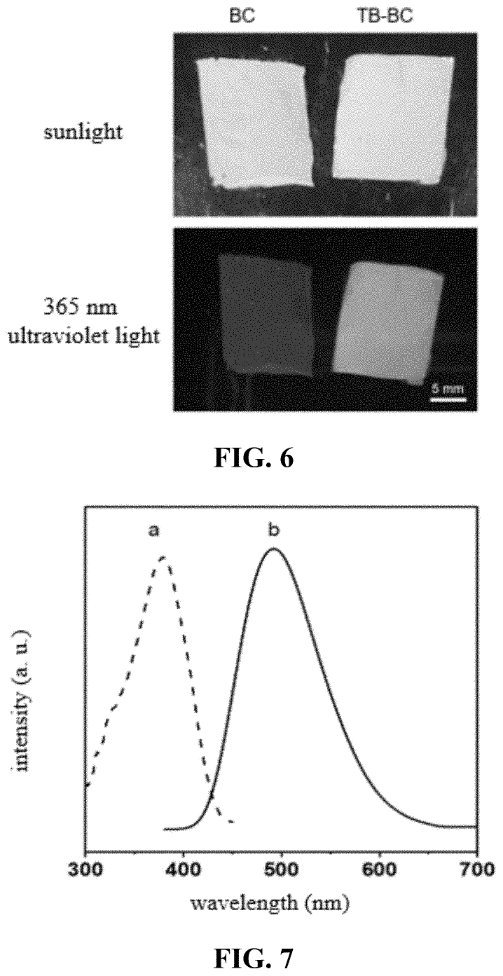

FIG. 6 is a picture of TB-BC/PVP electrospun polymer fiber membrane and PVP electrospun polymer fiber membrane under sunlight and 365 nm ultraviolet light. It can be seen from FIG. 6 that under sunlight, there is no significant difference in appearance between PVP electrospun polymer fiber membrane and TB-BC/PVP electrospun polymer fiber membrane; under ultraviolet light, PVP electrospun polymer fiber membrane has no fluorescence, TB-BC/PVP electrospun polymer fiber membrane has obvious yellow fluorescence, indicating that the aggregation-induced emission polymer synthesized in the present disclosure can be used for electrospinning to synthesize electrospinning film, and can be applied to the production of fluorescent patterns.

### Comparative Example 2

The basal medium was sterilized at 115° C. for 20-30 min and cooled to room temperature to obtain a sterilized basal medium; the composition of the basal medium was: 25 g/L glucose, 5 g/L yeast extract, 5 g/L peptone, 1.2 g/L citric acid, 2.7 g/L disodium hydrogen phosphate and deionized water.

A single colony of Acetobacter xylinum was inoculated into 20 mL of sterilized basal medium and cultured at 30° C. for 24 h to obtain the Acetobacter xylinum seed solution.

The Acetobacter xylinum seed solution accounting for 7% of the volume of the basal medium was inoculated in a sterilized basal medium, and cultivated in a constant temperature incubator at 30° C. for 4-5 d. After rinsing with distilled water to remove the basal medium and impurities on the surface, it was placed in a sodium hydroxide solution with a concentration of 0.5 mol/L and subjected to alkali treatment at 60° C. for 12 h to remove bacterial protein and residual basal medium, cooled to room temperature, fully washed with distilled water until the pH of the washing solution was neutral, and vacuum dried to constant weight at 30° C. to obtain the polymer having the structure represented by formula III (bacterial cellulose, abbreviated as BC).

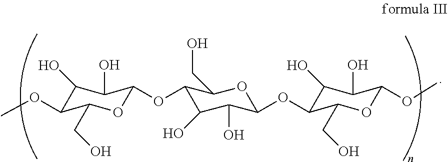

### Comparative Example 3

Aggregation-induced emission polymer synthesized by physical immersion method

The aggregation-induced emission small molecule monomer having the structure represented by formula IIa was dissolved in dimethyl sulfoxide to obtain an aggregation-induced emission small molecule solution with a concentration of 10 μg/mL.

The polymer BC prepared in Comparative Example 2 was placed in the aggregation-induced emission small molecule solution for 5 d at 37° C., rinsed with distilled water to remove impurities on the surface, and placed in sodium hydroxide solution with a concentration of 0.5 mol/L, and subjected to alkali treatment at 60° C. for 12 h to remove bacterial protein, cooled to room temperature, fully washed with distilled water until the pH of the washing solution was neutral, and vacuum dried at 30° C. to a constant weight to obtain aggregation-induced emission polymer.

### Comparative Example 4

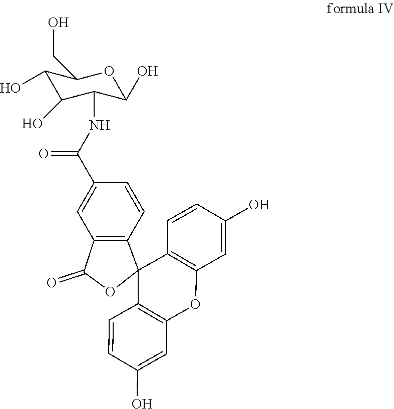 [SMILES: O=C(NC1C(O)OC(CO)C(O)C1O)c1ccc2c(c1)C(=O)OC21c2ccc(O)cc2Oc2cc(O)ccc21]

The aggregation-caused quenching polymer (5CF-BC) was prepared according to the method of Example 3. The difference from Example 3 is that the molecule monomer is used to replace the aggregation-induced emission small molecule monomer. The aggregation-caused quenching small molecular monomer has the structure represented by formula IV, and the synthesis route is shown in the literature (A natural in situ fabrication method of functional bacterial cellulose using a microorganism. Nat. Commun. 2019, 10, 437) formula IV, and finally the target product is obtained.

### Test Example

(1) Fluorescence Performance Test

The BC, 5CF-BC, TPE-BC and TB-BC were dissolved in THF to prepare a polymer solution with a concentration of 1 mg/mL, and the fluorescence spectrum of the polymer solution was measured with a fluorometer.

FIG. 7 shows the fluorescence excitation and emission spectrum of TPE-BC, where a is the excitation spectrum and b is the emission spectrum; it can be seen from FIG. 7 that the Stokes shift of TPE-BC is 125 nm.

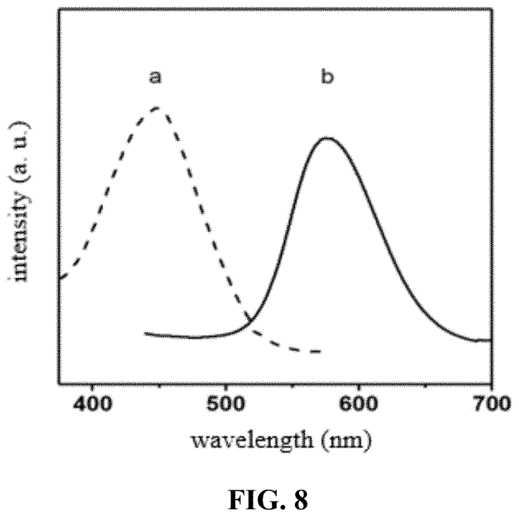

FIG. 8 shows the fluorescence excitation and emission spectrum of TB-BC, where a is the excitation spectrum and b is the emission spectrum. It can be seen from FIG. 8 that the Stokes shift of TB-BC is 130 nm.

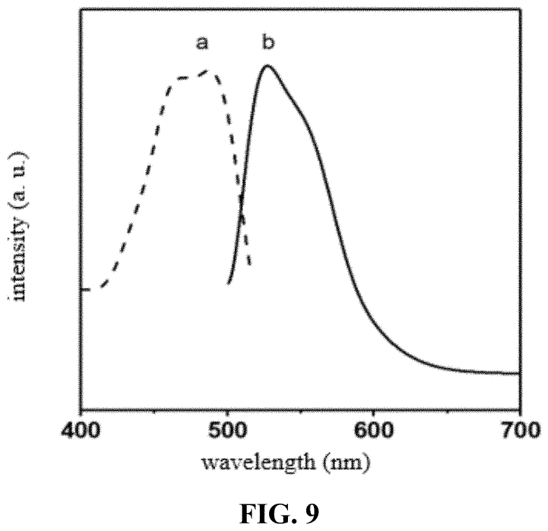

FIG. 9 is the fluorescence excitation and emission spectrum of 5CF-BC, where a is the excitation spectrum and b is the emission spectrum. It can be seen from FIG. 9 that the Stokes shift of 5CF-BC is about 40 nm.

It shows that the Stokes shift of the functionalized BC molecule TPE-BC and TB-BC with the AIE effect is much larger than the Stokes shift of the functionalized BC molecule 5CF-BC with the ACQ effect; wherein, Stokes shift=wavelength at the peak of the emission spectrum-wavelength at the peak of the excitation spectrum.

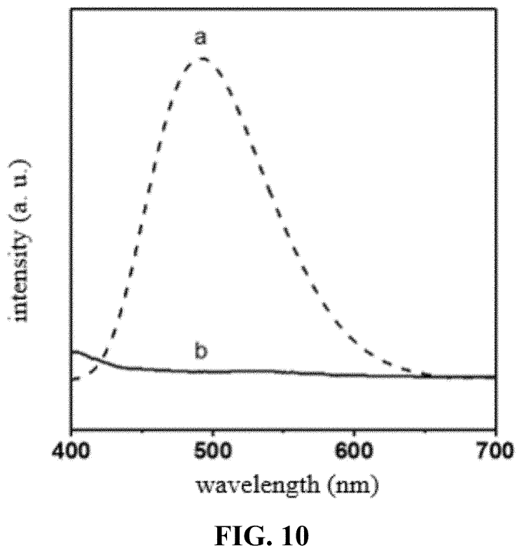

FIG. 10 shows the fluorescence spectra of TPE-BC and BC, where a is TPE-BC and b is BC. It can be seen from FIG. 10 that TPE-BC has an obvious fluorescence emission peak at 500 nm, while BC has no fluorescence emission peak at 500 nm and no fluorescence is generated.

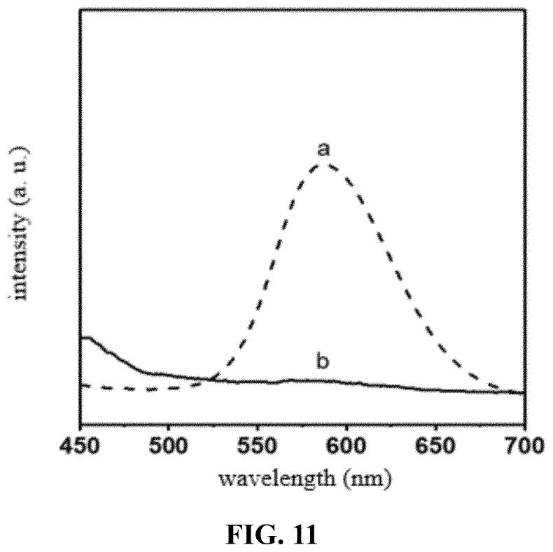

FIG. 11 shows the fluorescence spectra of TB-BC and BC, where a is TB-BC and b is BC. It can be seen from FIG. 11 that TB-BC has an obvious fluorescence emission peak at 570 nm, while BC has no fluorescence emission peak at 570 nm and no fluorescence is generated; it shows that the preparation method provided by the present disclosure successfully synthesizes a fluorescent aggregation-induced emission polymer.

(2) Laser Confocal Microscope Test

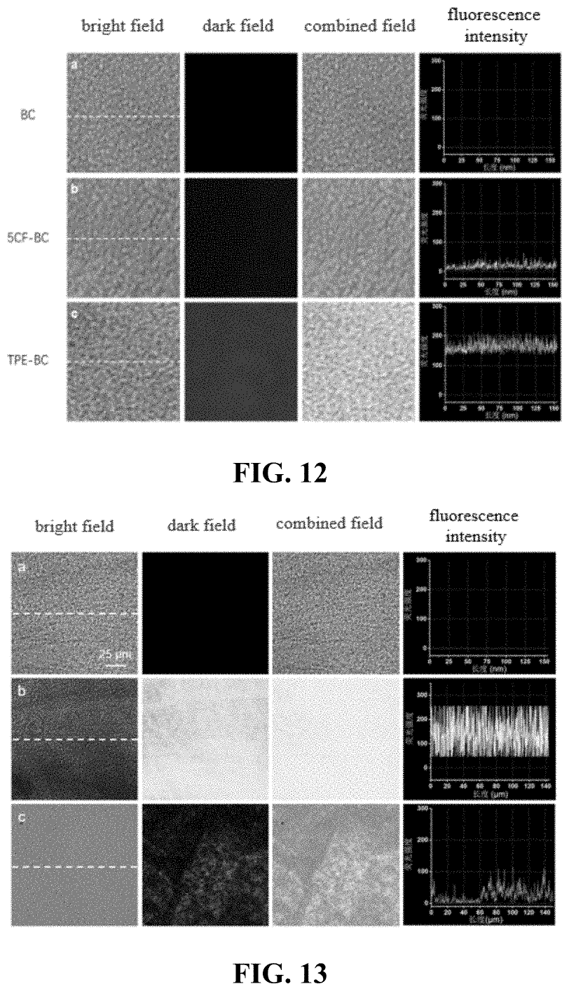

The BC, TPE-BC, TB-BC, TB/BC and 5CF-BC were tested by laser confocal microscope. The test results are shown in FIG. 12-13. FIG. 12 is a confocal laser microscope image of BC, 5CF-BC, and TPE-BC, where a is BC, b is 5CF-BC, and c is TPE-BC. It can be seen from FIG. 12 that BC has no fluorescence, TPE-BC has strong and uniform cyan-blue fluorescence, and 5CF-BC has weak cyan-blue fluorescence. It shows that polymers synthesized with small molecules with AIE effect have stronger fluorescence than polymers synthesized with small molecules with ACQ effect.

FIG. 13 is a confocal laser microscope image of BC, TB/BC and TB-BC, where a is BC, b is TB/BC, and c is TB-BC. It can be seen from FIG. 13 that BC has no fluorescence, TB-BC has a strong and uniform orange-yellow fluorescence, and TB/BC has a weak and uneven orange-yellow fluorescence. The aggregation-induced emission polymer synthesized by the physical immersion method was observed to have uneven fluorescence distribution under a laser confocal microscope, while the fluorescence distribution of the biosynthesized aggregation-induced emission polymer was even observed under a laser confocal microscope.

(3) Solubility Test

The solubility test of TB-BC and BC was carried out. 1 mg of TB-BC and BC were dissolved in 1 mL of tetrahydrofuran, respectively, and subjected to ultrasonic treatment under 400 W at room temperature for 3 min. The TB-BC was completely dissolved, but the BC was not dissolved. It indicates that the aggregation-induced emission polymer prepared in the present disclosure can be dissolved in tetrahydrofuran.

(4) Scanning Electron Microscope

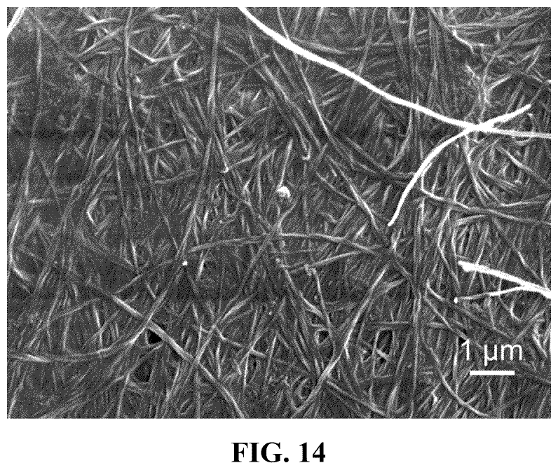

The TB-BC was characterized by scanning electron microscopy, and the results are shown in FIG. 14. It can be seen from FIG. 14 that the aggregation-induced emission polymer prepared by the present disclosure is composed of fibers with a diameter of 100 nm.

The above are only the preferred embodiments of the present disclosure. It should be pointed out that for those of ordinary skill in the art, without departing from the principle of the present disclosure, several improvements and modifications can be made, and these improvements and modifications should also be regarded as the protection scope of the present disclosure.

What is claimed is:

1. An aggregation-induced emission polymer, wherein having a structure represented by formula I: 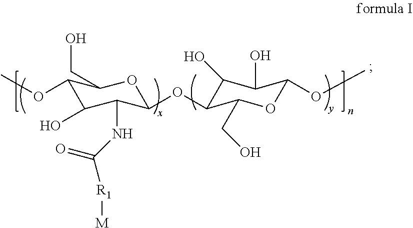
In the formula I, R₁ includes any one of the following structures: 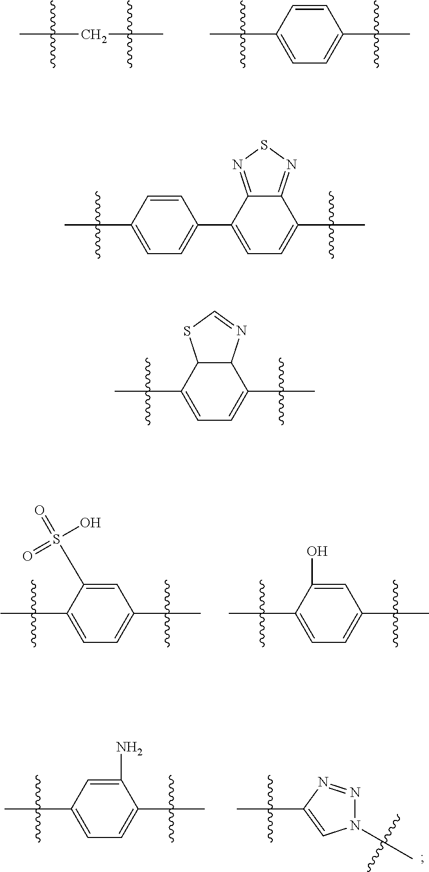
In the formula I, M has the structure represented by formula M-a or formula M-b: 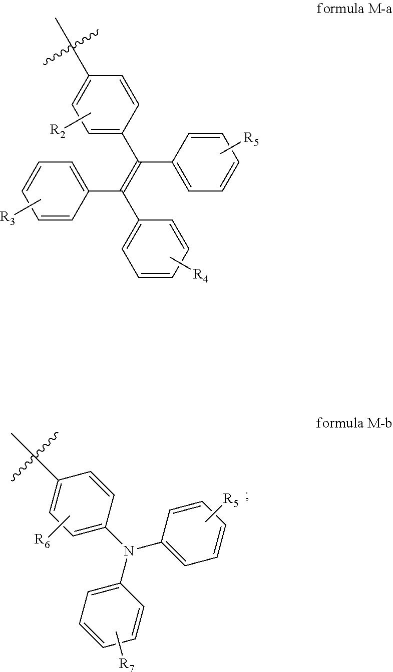
R₂-R₅ in the formula M-a and R₆-R₈ in the formula M-b independently include any one of the following structures: 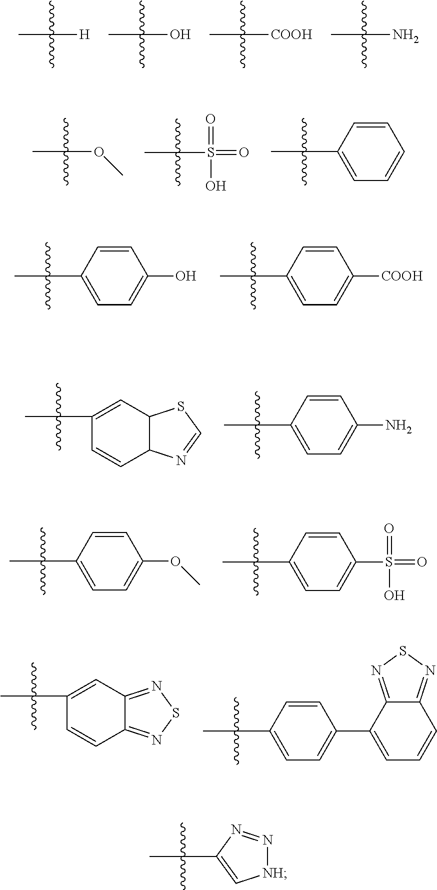
n is 1500-6000.

2. A method for preparing the aggregation-induced emission polymer according to claim 1, wherein comprising the following steps:
Placing an aggregation-induced emission small molecule monomer in a basal medium, then inoculating a bacterial seed solution, and culturing the obtained reaction solution to obtain an aggregation-induced emission polymer;
The aggregation-induced emission small molecule monomer has a structure represented by formula II: 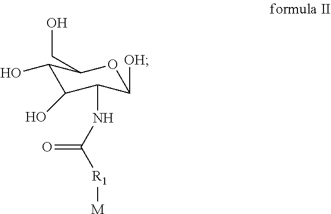
In the formula II, R₁ includes any one of the following structures: 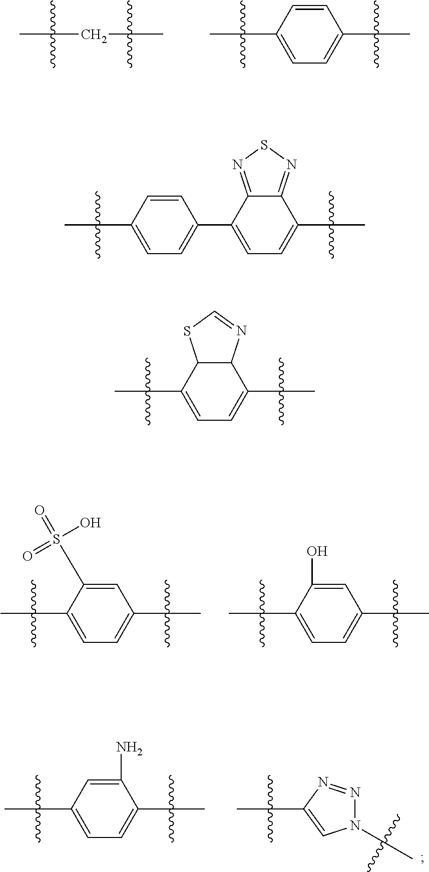
In the formula II, M has the structure represented by formula M-a or formula M-b: 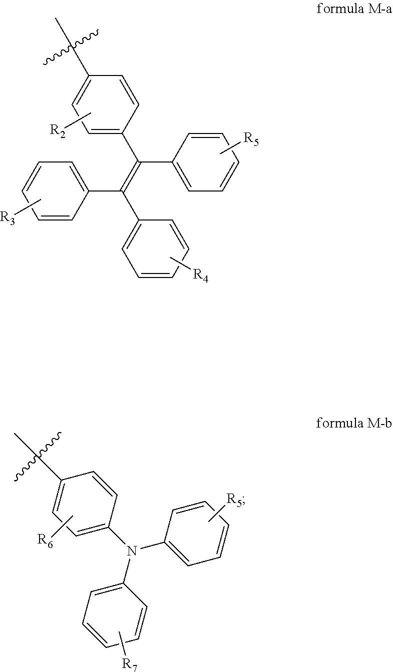
R₂-R₅ in the formula M-a and R₆-R₈ in the formula M-b independently include any one of the following structures:

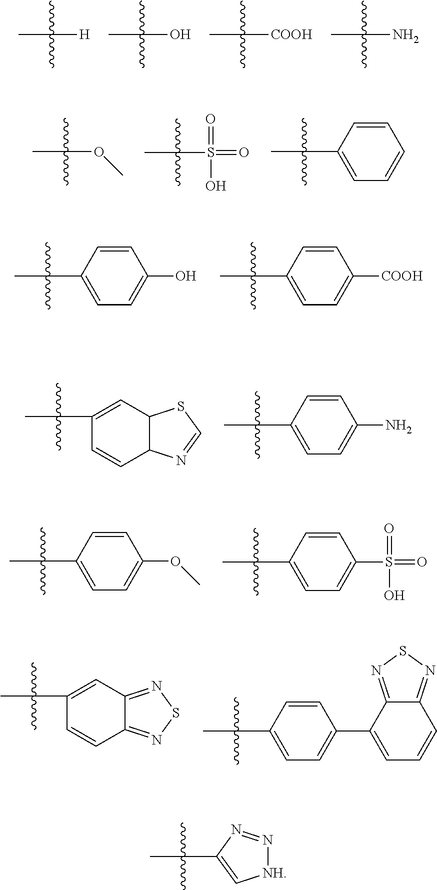

3. The preparation method according to claim 2, wherein the aggregation-induced emission small molecule monomer preferably has a structure represented by formula IIa, formula Ilb or formula IIc:

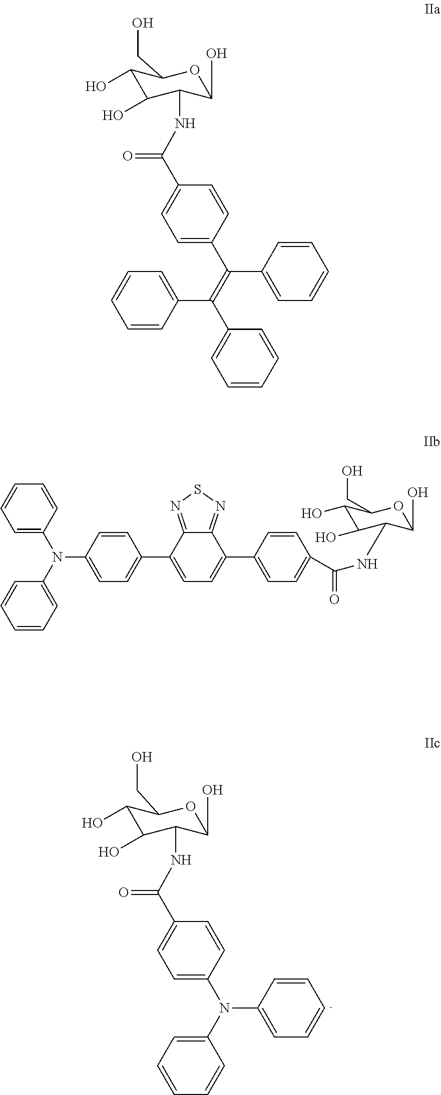 [SMILES: O=C(NC1C(O)OC(CO)C(O)C1O)c1ccc(C(=C(c2ccccc2)c2ccccc2)c2ccccc2)cc1 ; O=C(NC1C(O)OC(CO)C(O)C1O)c1ccc(-c2ccc(-c3ccc(N(c4ccccc4)c4ccccc4)cc3)c3nsnc23)cc1 ; O=C(NC1C(O)OC(CO)C(O)C1O)c1ccc(N(c2ccccc2)c2ccccc2)cc1]

4. The preparation method according to claim 2, wherein the concentration of the aggregation-induced emission small molecule monomer in the reaction solution is 0.001-1 mg/mL.

5. The preparation method according to claim 3, wherein the concentration of the aggregation-induced emission small molecule monomer in the reaction solution is 0.001-1 mg/mL.

6. The preparation method according to claim 2, wherein the chemical composition of the basal medium comprises: 20-30 g/L glucose, 4-6 g/L yeast extract, 4-6 g/L peptone, 1.1-1.3 g/L citric acid, 2.3-2.9 g/L disodium hydrogen phosphate and water.

7. The preparation method according to claim 2, wherein the inoculum size of the bacterial seed solution is 1-50% of the volume of the medium; and the bacterial cell density (OD₆₀₀) of the bacterial seed solution is 0.6-1.2.

8. The preparation method according to claim 6, wherein the inoculum size of the bacterial seed solution is 1-50% of the volume of the medium; and the bacterial cell density (OD₆₀₀) of the bacterial seed solution is 0.6-1.2.

9. The preparation method according to claim 2, wherein the temperature of the culture is 20-45° C., and the time is 2-8 d.

10. Application of the aggregation-induced emission polymer according to claim 1 in light-emitting diodes, bioimaging, fluorescent films, biosensors or chiral separations.
